(19) United States
(12) Patent Application Publication
(10) Pub. No.: US 2024/0390719 A1
(43) Pub. Date: Nov. 28, 2024

(54) SAFEGUARD AGENT AND USE THEREOF

(71) Applicant: Haibo Li, Shanghai (CN)

(72) Inventor: Haibo Li, Shanghai (CN)

(21) Appl. No.: 18/672,155

(22) Filed: May 23, 2024

Related U.S. Application Data
(60) Provisional application No. 63/468,811, filed on May 25, 2023.

(51) Int. Cl. A62D 1/00; C07C 19/08; C07C 19/14; C07C 19/16; C07C 21/18; C07C 43/04; H01M 10/0525; H01M 50/103; H01M 50/105; H01M 50/107; H01M 50/383; H01M 50/673

(52) U.S. Cl. CPC A62D 1/0057; C07C 19/08; C07C 19/14; C07C 19/16; C07C 21/18; C07C 43/04; H01M 10/0525; H01M 50/103; H01M 50/105; H01M 50/107; H01M 50/383; H01M 50/673

(57) ABSTRACT

The present disclosure discloses a safeguard agent and a use thereof. The present disclosure provides a safeguard agent consisting of component A and component B, and the molar ratio of the component A to the component B is 1:(1-4). The safeguard agent in the present disclosure can cool the protected space and heat sources, physically isolate heat sources, prevent burning or extinguish the flame in the space, suppress explosion, and effectively and continuously suppress and prevent reignition. Using the safeguard agent of the present disclosure as a suppression agent for battery thermal runaway can achieve the purpose of terminating thermal runaway after cooling, antiflaming, suppression, or extinguishing of a battery in thermal runaway, and more batteries can achieve constant voltage and good appearance without damage phenomena. For other batteries affected by heat in the space, the safeguard agent can also effectively prevent or suppress potential thermal runaway.

### CROSS-REFERENCE TO RELATED APPLICATION

The present application claims priority from U.S. Provisional Patent Application No. 63/468,811 filed on May 25, 2023, the contents of which are incorporated herein by reference in their entirety.

### TECHNICAL FIELD

The present disclosure relates to a safeguard agent and a use thereof, which are used for the suppression and safety protection of battery thermal runaway.

### BACKGROUND

There are two main technologies for preventing burning, i.e., fire retardants and extinguishants, wherein the former renders burning impossible or difficult to occur, and the latter releases agents to extinguish the flame when the burning occurs. Fire retardants are added to combustibles, such as treating fabrics, wood, plastics, and rubber, so that combustibles cannot burn, or cannot burn constantly at high temperatures. Or, fire-retardant glue or fire-retardant board is used to physically isolate the burning material, making it difficult for the flame to spread and the burning to continue. The extinguishant is stored in an independent container. Once a fire starts, it is artificially or automatically sprayed to the object or the room on fire to extinguish the flame and the burning.

Fire retardants are usually added to combustibles as additives. However, for some high-risk commodities such as lithium batteries, even with the addition of fire retardants, thermal runaway and burning still occur inevitably, and explosions also occur from time to time. In addition, adding a large amount of fire retardants to the battery will affect the electrochemical performance of the battery, and the economic efficiency is poor.

Liquid water and foam, chemical gases and solid dry powder are generally used as extinguishants, and local application and total flooding are used to extinguish the fire. Local application refers to spraying an extinguishant on the protective object for a long time, without the need for space limitation, such as extinguishing with an extinguisher and spraying pressure water mist on fire. Total flooding refers to gaseous extinguishants, such as heptafluoropropane FM-200, perfluorohexanone Novec-1230, inert gas IG-541, which require a well-sealed protected space. It is necessary to test the enclosure integrity and leakage of the protected space according to relevant standards and procedures, so that a certain volume percentage and a certain duration of chemical concentration can be maintained in the space in order to extinguish the flame and constantly suppress the burning.

For small or micro spaces, such as battery cabinets or battery packages, conventional technology of chemical gaseous extinguishants cannot effectively deal with the situation which the enclosure integrity is damaged, and the agent will leak or diffuse quickly and the purpose of fire extinguishing cannot be achieved. The thermal runaway of a battery is caused by internal short circuit brought by internal factors such as battery manufacturing defects or lithium dendrite phenomenon, or external factors such as impact, heating, external short circuit or wiring process. After the thermal runaway of the battery, an irreversible energy release is formed, manifested as constant heat generation, generation of inflammable and explosive gas, violent deflagration or explosion, continuous burning, smoke generation, or flameless combustion. Most of these phenomena appear in combination. Thermal runaway is not equal to burning, and burning is only one of the accompanying phenomena. Thermal runaway without burning, only with heat and gas generation, is also more common. Thermal runaway sometimes manifests itself as flameless combustion, such as the ternary pouch battery added with fire retardants. Flameless combustion is live coal-like glow and smoke, which is a typical characteristic of deep-seated fire.

It'd been confirmed by a large number of experiments and research reports that conventional technologies of fire retardants and extinguishants, commonly used extinguishants such as perfluorohexanone, heptafluoropropane, hexafluoropropane, aerosol, high-pressure water mist, low-expansion foam and high-expansion foam, all cannot effectively deal with thermal runaway and burning of lithium battery in a small amount. After the fire is extinguished in battery thermal runaway, it is normal for the battery to resume burning because the interior of the battery constantly generates flammable gas and heat. For application of fire fighting such as water and foam, a large amount of agents form water damages, which results in secondary losses and total battery loss. Studies and tests on heptafluoropropane and Novec-1230, etc. have shown that only surface flame can be extinguished, and even if the used amount of the agents is huge, the thermal runaway of battery still cannot be terminated. 3M, the manufacturer of Novec-1230, had stated in a written declaration in August 2017 that clean agents are ineffective in avoiding and preventing thermal runaway, as are foams and dry powders.

It has been known to the public that lithium batteries are prone to thermal runaway and reignition frequently, for which the existing flame-retardant and fire-fighting technologies are powerless. The strong market demand urgently requires new forms of products to solve the above safety problems, and creative solutions and technical routes are required.

### BRIEF SUMMARY OF THE INVENTION

Therefore, the technical problem solved by the present disclosure is a problem that if a large number of batteries are in an enclosure, or a space where the enclosure integrity is damaged or a space with poor sealing space, the batteries are prone to thermal runaway which cannot be terminated, and the batteries catch fire after the thermal propagation, and reignition after being extinguished by fire-fighting technology. The present disclosure provides a safeguard agent and a use thereof. The safeguard agent of the present disclosure can continuously cool, physically isolate and chemically suppress the burning to prevent the formation of the burning conditions, terminate the chemical reaction or suppress the unfavorable chain reaction, and finally heat and flammable and explosive gases are no longer produced, and completely terminate thermal runaway of a battery when the thermal runaway of the battery generates a large amount of heat and gas.

Specifically, when the signs of thermal runaway appear, the safeguard agent of the present disclosure can be released at the same time or after a delay, and it can remain for a long time even in a space with poor sealing, and continue to play the role of cooling, isolation and suppression, preventing burning or suppressing explosions. Even if the burning occurs before release, it can extinguish the flame immediately and suppress it for a long time to prevent reignition. As a result, the following logical evolution is fundamentally blocked: When the thermal runaway of the battery cannot be terminated, thermal propagation occurs between the batteries, and the battery is thermally runaway again, further igniting and burning.

The present disclosure provides a safeguard agent, consisting of component A and component B; the component A is selected from one or more of a C₅-C₈ perfluoroalkane, a C₅-C₇ perfluorocycloalkane which is unsubstituted or substituted by one or more trifluoromethyl groups, a C₅-C₈ fluoroalkane containing 1, 2, 3 or 4 hydrogen atoms (the C₅-C₈ fluoroalkane containing 1, 2, 3 or 4 hydrogen atoms consists of three elements fluorine, carbon and hydrogen), a C₄-C₇ perfluoroketone, a C₄-C₇ fluoroether, a C₂-C₆ perfluorocyclic ether which is unsubstituted or substituted by one or more C₁-C₃ perfluoroalkyl groups and heptafluoro-2-(1,2,2-trifluoroethyleneoxy)propane; the C₄-C₇ fluoroether contains at least 7 fluorine atoms;
the component B is a C₃-C₅ bromofluoroalkene and/or a C₂-C₄ iodofluoroalkane; the bromofluoroalkene contains at least 2 fluorine atoms; the C₂-C₄ iodofluoroalkane contains at least 4 fluorine atoms;
the molar ratio of the component A to the component B is 1:(1-4).

In a certain embodiment, the safeguard agent is a cooling suppressant or an extinguishant.

In a certain embodiment, the safeguard agent can be used in a walled space, a sealed space or an enclosure, such as the walled space or the enclosure.

In a certain embodiment, the safeguard agent is used to protect a battery or suppress energy that is continuously released from the battery during thermal runaway.

In a certain embodiment, the battery is preferably a battery that releases energy in seconds when thermal runaway occurs, preferably a lithium-ion battery that releases energy in seconds.

In a certain embodiment, the lithium-ion battery is a ternary lithium battery, a lithium-cobalt battery or a lithium manganese battery.

In a certain embodiment, the lithium-ion battery is a prismatic battery, a pouch battery or a cylinder battery.

In a certain embodiment, the release mode of energy release during thermal runaway is violent heat generation, gas generation, burning or explosion; the thermal runaway is caused by internal short circuit or external short circuit of the battery, which can be simulatively induced through nail penetration or heating experiment.

In a certain embodiment, the safeguard agent is a homogeneous liquid (wherein the meaning of homogeneous liquid is that the components of the safeguard agent are homogeneously distributed without stratification).

In a certain embodiment, the safeguard agent can be used to suppress heat generation, gas generation or burning caused by thermal runaway of a battery.

In one embodiment, the molar ratio of the component A to the component B is 1:(2-4), such as 1:3.

In a certain embodiment, the C₂-C₄ iodofluoroalkane contains one iodine atom.

In a certain embodiment, the C₃-C₅ bromofluoroalkene contains one bromine atom.

In a certain embodiment, the C₅-C₈ perfluoroalkane can be selected from perfluorohexane and/or perfluoroheptane, and the perfluorohexane is preferably perfluoro-n-hexane, perfluoro-2-methylpentane or perfluoro-2,3-dimethylbutane; the perfluoroheptane is preferably perfluoro-n-heptane. The C₅-C₈ perfluoroalkane is, for example, selected from one or more of

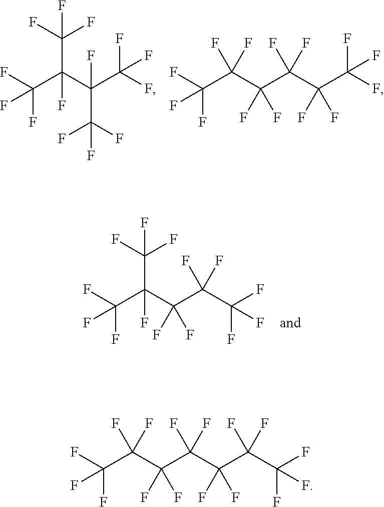

In a certain embodiment, the C₅-C₈ fluoroalkane containing 1, 2, 3 or 4 hydrogen atoms can be decafluoropentane and/or monohydrotridecafluorohexane, such as

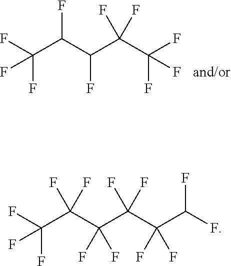 [SMILES: FC(C(F)C(F)(F)C(F)(F)F)C(F)(F)F ; FC(F)C(F)(F)C(F)(F)C(F)(F)C(F)(F)C(F)(F)F]

In a certain embodiment, the C₅-C₇ perfluorocycloalkane which is unsubstituted or substituted by one or more trifluoromethyl groups can be dodecafluorocyclohexane and/or perfluoromethylcyclopentane.

In a certain embodiment, the C₄-C₇ perfluoroketone contains 1 or 2 carbonyl groups.

In a certain embodiment, the C₄-C₇ perfluoroketone is 1,1,1,3,4,4,4-heptafluoro-3-(trifluoromethyl)butan-2-one and/or perfluoro-2-methyl-3-pentanone.

In a certain embodiment, the C₄-C₇ fluoroether contains 1 or 2 oxygen atoms.

In a certain embodiment, the C₄-C₇ fluoroether can be selected from one or more of 2-(difluoro(methoxy)methyl)-1,1,1,2,3,3,3-heptafluoropropane, 2,2,3,3,3-pentafluoropropyl difluoromethyl ether, methylnonafluorobutylether, heptafluoropropyl-1,2,2,2-tetrafluoroethyl ether, 1,1,1,2,2,3,3-heptafluoro-3-methoxypropane, 2-(difluoromethoxymethyl)-1,1,1,2,3,3,3-heptafluoropropane and 1-ethoxy-1,1,2,3,3,3-hexafluoro-2-(trifluoromethyl)propane; for example, selected from one or more of

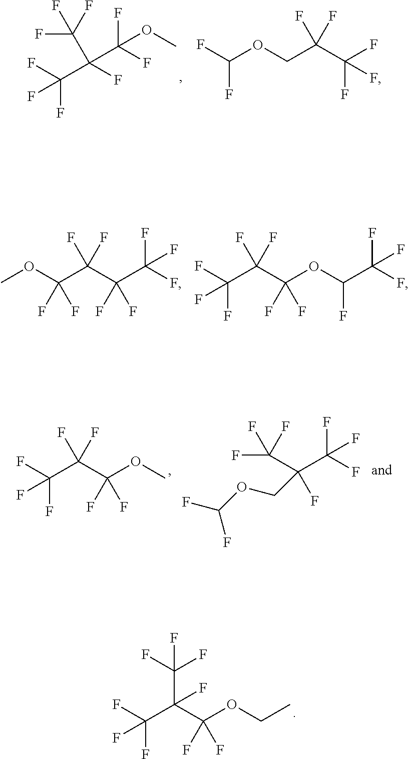

In a certain embodiment, the C₂-C₆ perfluorocyclic ether which is unsubstituted or substituted by one or more C₁-C₃ perfluoroalkyl groups contains 1 oxygen atom.

In a certain embodiment, the C₂-C₆ perfluorocyclic ether which is unsubstituted or substituted by one or more C₁-C₃ perfluoroalkyl groups, wherein the C₂-C₆ refers to the carbon atoms on the ring.

In a certain embodiment, the C₂-C₆ perfluorocyclic ether which is unsubstituted or substituted by one or more C₁-C₃ perfluoroalkyl groups can be perfluorocycloheptyl ether or perfluoro-2-methyl-2,3-epoxypentane.

In a certain embodiment, the C₃-C₅ bromofluoroalkene can be selected from one or more of 3-bromo-3,3-difluoropropene, 2-bromo-3,3,3-trifluoropropene, 3-bromo-1,1,3,3-tetrafluoropropene, 2-bromo-3,3,4,4,4-pentafluoro-1-butene, 2-bromo-3,4,4,4-tetrafluoro-3-(trifluoromethyl)but-1-ene and 2-bromo-3,3,4,4,5,5,5-octafluoro-1-pentene, for example, selected from one or more of

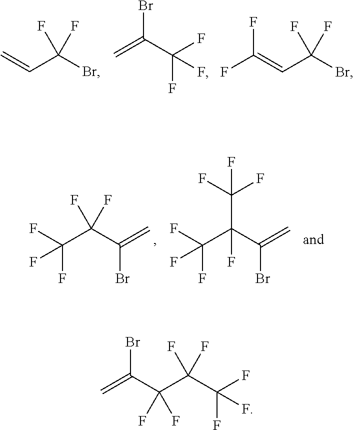

In a certain embodiment, the C₂-C₄ iodofluoroalkane can be selected from one or more of 1,1,1,2-tetrafluoro-2-iodoethane, iodo-1,1,2,2-tetrafluoroethane and iodoperfluorobutane, the iodoperfluorobutane is preferably 1-iodoperfluorobutane and/or 2-iodononafluorobutane, for example, selected from one or more of

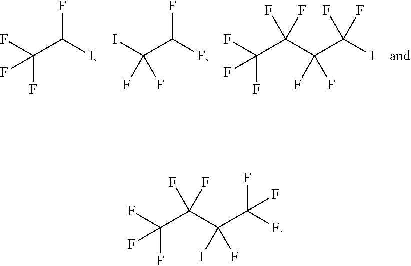

In a certain embodiment, the component A is selected from one or two of a C₅-C₇ perfluorocycloalkane which is unsubstituted or substituted by one or more trifluoromethyl groups, a C₄-C₇ perfluoroketone, C₂-C₄ perfluorocyclic ether which is unsubstituted or substituted by one or more C₁-C₃ perfluoroalkyl groups and a C₄-C₇ fluoroether, the C₄-C₇ fluoroether contains at least 7 fluorine atoms.

In a certain embodiment, the component A is selected from one or more of

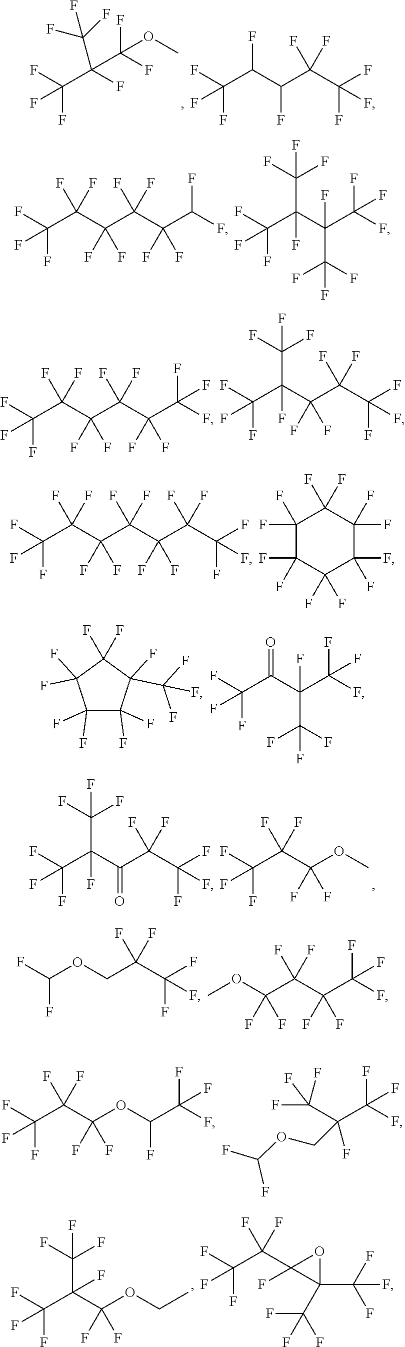

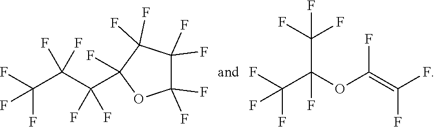 [SMILES: FC(F)(F)C(F)(F)C(F)(F)C1(F)OC(F)(F)C(F)(F)C1(F)F ; FC(F)=C(F)OC(F)(C(F)(F)F)C(F)(F)F]

In a certain embodiment, the component A is selected from one or two of

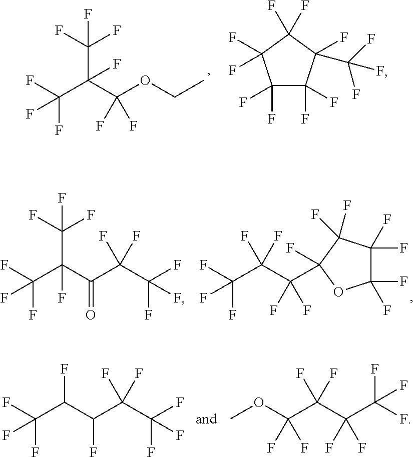

In a certain embodiment, the component B is preferably selected from a C₃ bromofluoroalkene and/or a C₄ iodoperfluoroalkane; for example, selected from one or more of

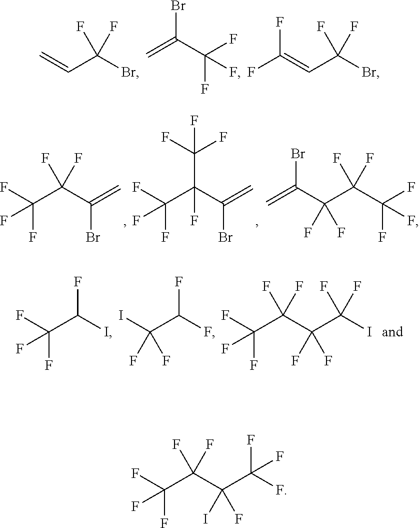

In a certain embodiment, the component B is more preferably selected from

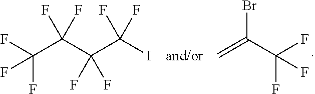

In a certain embodiment, the boiling point of the component A can be 20-85° C., preferably 25-60° C., such as 48° C., 55° C., 49.2° C., 49.5° C., 60° C. or 72° C.

In a certain embodiment, the boiling point of the component B can be 30-80° C., preferably 30-67° C., such as 34° C. or 64-67° C.

In a certain embodiment, the molecular weight of the component A can be 200-400 g/mol, preferably 250-370 g/mol, such as 250 g/mol, 252 g/mol, 366 g/mol, 264 g/mol, 300 g/mol or 316 g/mol.

In a certain embodiment, the molecular weight of the component B can be 150-400 g/mol, preferably 170-350 g/mol, such as 175 g/mol or 346 g/mol.

In a certain embodiment, the latent heat of vaporization of the component A can be 21.6-34.4 kJ/mol, preferably 26-32 kJ/mol, such as 27.3 kJ/mol, 27.8 kJ/mol, 27.9 kJ/mol, 28.3 kJ/mol, 30.7 kJ/mol or 31.5 kJ/mol.

In a certain embodiment, the latent heat of vaporization of the component B can be 24.2-33.2 kJ/mol, preferably 28-33 kJ/mol, such as 30.6 kJ/mol or 29.6 kJ/mol.

In a certain embodiment, the safeguard agent adopts scheme 1, scheme 2, scheme 3, scheme 4, scheme 5, scheme 6, scheme 7 or scheme 8

| | | | Molar ratio of |
|---|---|---|---|
| | | | component A to |
| | Component A | Component B | component B |
| Scheme 1 | 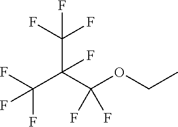 [SMILES: CCOC(F)(F)C(F)(C(F)(F)F)C(F)(F)F] | 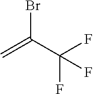 | 1:3 |
| Scheme 2 | 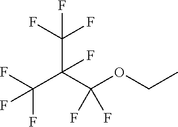 [SMILES: CCOC(F)(F)C(F)(C(F)(F)F)C(F)(F)F] | 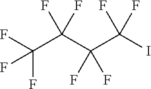 [SMILES: FC(F)(F)C(F)(F)C(F)(F)C(F)(F)I] | 1:2 |
| Scheme 3 | 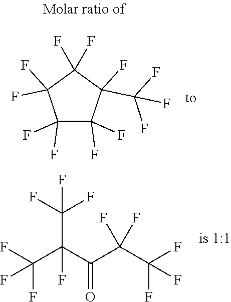 | 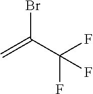 | 2:5 |
| Scheme 4 | 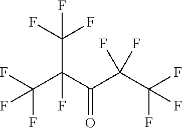 [SMILES: O=C(C(F)(F)C(F)(F)F)C(F)(C(F)(F)F)C(F)(F)F] | 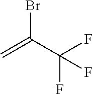 | 1:3 |
| Scheme 5 | 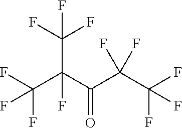 [SMILES: O=C(C(F)(F)C(F)(F)F)C(F)(C(F)(F)F)C(F)(F)F] | 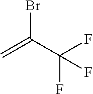 | 1:2 |
| Scheme 6 | 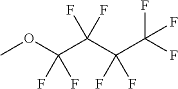 | 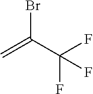 | 1:4 |
| Scheme 7 | 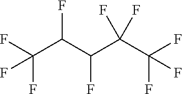 [SMILES: FC(C(F)C(F)(F)C(F)(F)F)C(F)(F)F] | 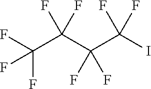 [SMILES: FC(F)(F)C(F)(F)C(F)(F)C(F)(F)I] | 1:1 |
| Scheme 8 | 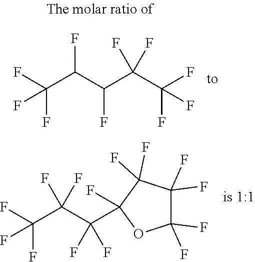 | 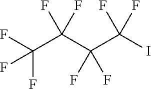 [SMILES: FC(F)(F)C(F)(F)C(F)(F)C(F)(F)I] | 1:2 |

The present disclosure also provides a safeguard agent, consisting of component A and component B; and the latent heat of vaporization of the component A is 21.6-34.4 kJ/mol; the latent heat of vaporization of the component B is 24.2-33.2 kJ/mol; the boiling point of the component A is 20-85° C.; the boiling point of the component B is 30-80° C.; the number of fluorine atoms of the compound in the component A is 7-16, and the number of carbon atoms of the compound in the component A is 4-7; the compound in the component B contains at least one bromine or iodine atom; the number of hydrogen atoms of the compound in the component A is 0-5; the molecular weight of the component A is 200-400 g/mol; the molecular weight of the component B is 150-400 g/mol;
the molar ratio of the component A to the component B is 1:(1-4).

In a certain embodiment, the molar ratio of the component A to the component B is 1:(2-4), such as 1:3.

In a certain embodiment, the component A is selected from one or more of a C₅-C₈ perfluoroalkane, a C₅-C₇ perfluorocycloalkane which is unsubstituted or substituted by one or more trifluoromethyl groups, a C₅-C₈ fluoroalkane containing 1, 2, 3 or 4 hydrogen atoms (the C₅-C₈ fluoroalkane containing 1, 2, 3 or 4 hydrogen atoms consists of three elements fluorine, carbon and hydrogen), a C₄-C₇ perfluoroketone, a C₄-C₇ fluoroether, a C₂-C₆ perfluorocyclic ether which is unsubstituted or substituted by one or more C₁-C₃ perfluoroalkyl groups and heptafluoro-2-(1,2,2-trifluoroethyleneoxy)propane; the C₄-C₇ fluoroether contains at least 7 fluorine atoms.

In a certain embodiment, the component B is a C₃-C₅ bromofluoroalkene and/or a C₂-C₄ iodofluoroalkane; the bromofluoroalkene contains at least 2 fluorine atoms; the C₂-C₄ iodofluoroalkane contains at least 4 fluorine atoms.

In one embodiment, the safeguard agent is a homogeneous liquid (wherein the meaning of homogeneous liquid is that the components of the safeguard agent are homogeneously distributed without stratification).

In a certain embodiment, the safeguard agent is a cooling suppressant or an extinguishant.

In one embodiment, the safeguard agent can be used in a walled space, a sealed space or an enclosure.

In a certain embodiment, the safeguard agent is used to protect a battery or suppress energy that is continuously released from the battery during thermal runaway.

In a certain embodiment, the battery is preferably a battery that releases energy in seconds when thermal runaway occurs, preferably a lithium-ion battery that releases energy in seconds.

In one embodiment, the lithium-ion battery is a ternary lithium battery, a lithium-cobalt battery or a lithium manganese battery.

In a certain embodiment, the lithium-ion battery is a prismatic battery, a pouch battery or a cylinder battery.

In a certain embodiment, the release mode of energy release during thermal runaway is violent heat generation, gas generation, burning or explosion; the thermal runaway can be caused by internal short circuit or external short circuit of the battery, which can be simulatively induced through nail penetration or heating experiment.

In a certain embodiment, the safeguard agent can be used to suppress heat generation, gas generation or burning caused by thermal runaway of a battery.

In a certain embodiment, the number of the release of the safeguard agent can be one or more times in the use.

In a certain embodiment, the more times are preferably three times in the use.

In a certain embodiment, the C₂-C₄ iodofluoroalkane contains one iodine atom.

In a certain embodiment, the C₃-C₅ bromofluoroalkene contains one bromine atom.

In a certain embodiment, the C₅-C₈ perfluoroalkane can be selected from perfluorohexane and/or perfluoroheptane, and the perfluorohexane is preferably perfluoro-n-hexane, perfluoro-2-methylpentane or perfluoro-2,3-dimethylbutane; the perfluoroheptane is preferably perfluoro-n-heptane. The C₅-C₈ perfluoroalkane is, for example, selected from one or more of

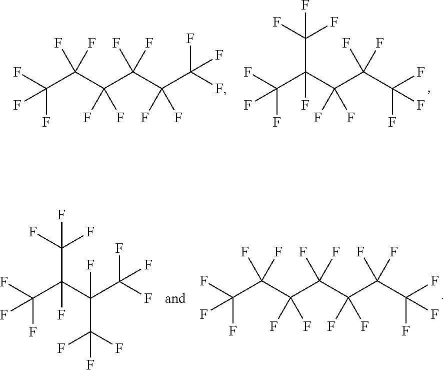

In a certain embodiment, the C₅-C₈ fluoroalkane containing 1, 2, 3 or 4 hydrogen atoms can be decafluoropentane and/or monohydrotridecafluorohexane, such as

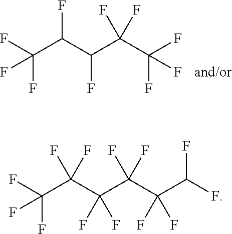 [SMILES: FC(C(F)C(F)(F)C(F)(F)F)C(F)(F)F ; FC(F)C(F)(F)C(F)(F)C(F)(F)C(F)(F)C(F)(F)F]

In a certain embodiment, the C₅-C₇ perfluorocycloalkane which is unsubstituted or substituted by one or more trifluoromethyl groups can be dodecafluorocyclohexane and/or perfluoromethylcyclopentane.

In a certain embodiment, the C₄-C₇ perfluoroketone contains 1 or 2 carbonyl groups.

In a certain embodiment, the C₄-C₇ perfluoroketone is 1,1,1,3,4,4,4-heptafluoro-3-(trifluoromethyl)butan-2-one and/or perfluoro-2-methyl-3-pentanone.

In a certain embodiment, the C₄-C₇ fluoroether contains 1 or 2 oxygen atoms.

In a certain embodiment, the C₄-C₇ fluoroether can be selected from one or more of 2-(difluoro(methoxy)methyl)-1,1,1,2,3,3,3-heptafluoropropane, 2,2,3,3,3-pentafluoropropyl difluoromethyl ether, methylnonafluorobutylether, heptafluoropropyl-1,2,2,2-tetrafluoroethyl ether, 1,1,1,2,2,3,3-heptafluoro-3-methoxypropane, 2-(difluoromethoxymethyl)-1,1,1,2,3,3,3-heptafluoropropane and 1-ethoxy-1,1,2,3,3,3-hexafluoro-2-(trifluoromethyl)propane; for example, selected from one or more of

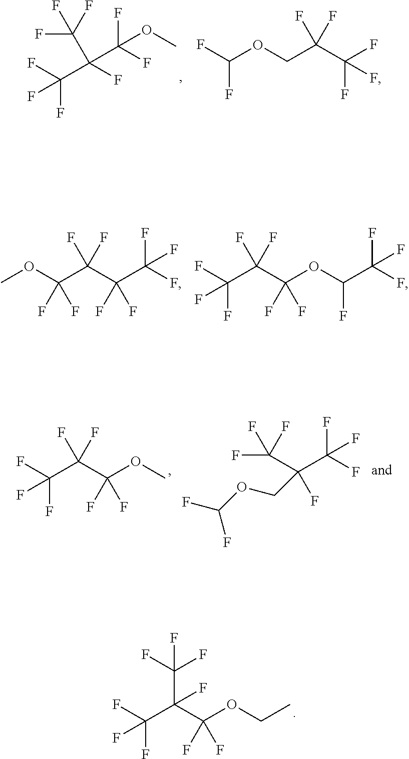

In a certain embodiment, the C₂-C₆ perfluorocyclic ether which is unsubstituted or substituted by one or more C₁-C₃ perfluoroalkyl groups contains one oxygen atom.

In a certain embodiment, the C₂-C₆ perfluorocyclic ether which is unsubstituted or substituted by one or more C₁-C₃ perfluoroalkyl groups can be perfluorocycloheptyl ether or 2-methyl-2,3-epoxypentane.

In a certain embodiment, the C₃-C₅ bromofluoroalkene can be selected from one or more of 3-bromo-3,3-difluoropropene, 2-bromo-3,3,3-trifluoropropene, 3-bromo-1,1,3,3-tetrafluoropropene, 2-bromo-3,3,4,4,4-pentafluoro-1-butene, 2-bromo-3,4,4,4-tetrafluoro-3-(trifluoromethyl)but-1-ene and 2-bromo-3,3,4,4,5,5,5-octafluoro-1-pentene, for example, selected from one or more of

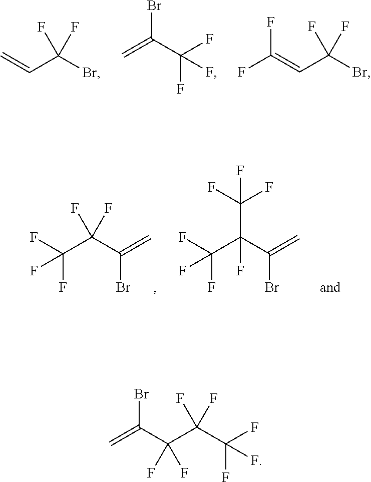

In a certain embodiment, the C₂-C₄ iodofluoroalkane can be selected from one or more of 1,1,1,2-tetrafluoro-2-iodoethane, iodo-1,1,2,2-tetrafluoroethane and iodoperfluorobutane, the iodoperfluorobutane is preferably 1-iodoperfluorobutane and/or 2-iodononafluorobutane, for example, selected from one or more of

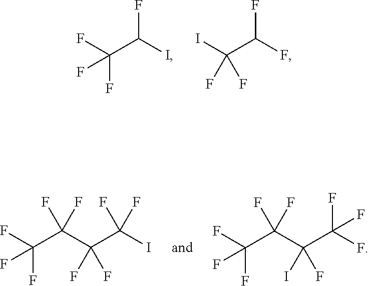

In a certain embodiment, the component A is selected from one or two of a C₅-C₇ perfluorocycloalkane which is unsubstituted or substituted by one or more trifluoromethyl groups, a C₄-C₇ perfluoroketone, a C₂-C₄ perfluorocyclic ether which is unsubstituted or substituted by one or more C₁-C₃ perfluoroalkyl groups and a C₄-C₇ fluoroether, the C₄-C₇ fluoroether contains at least 7 fluorine atoms.

In a certain embodiment, the component A is selected from one or more of

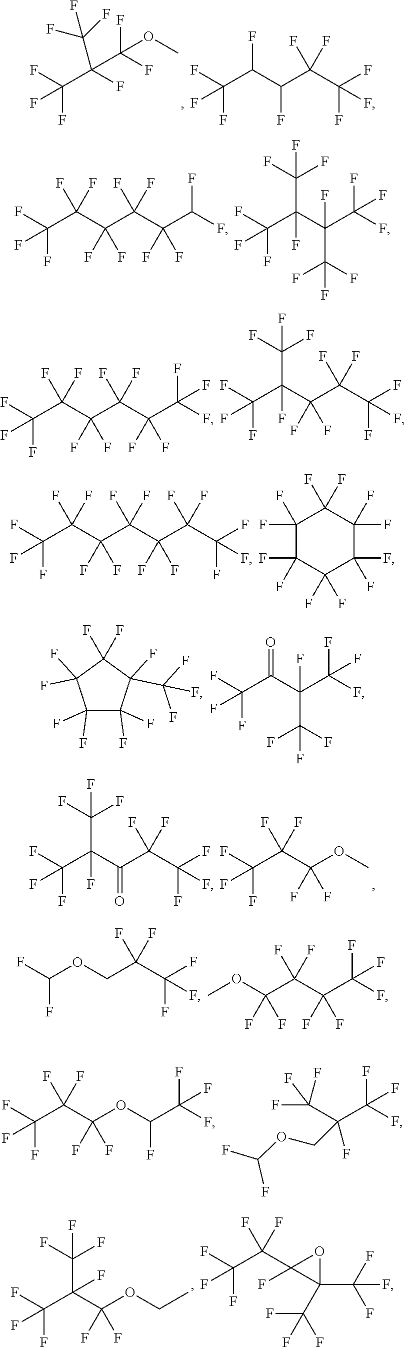

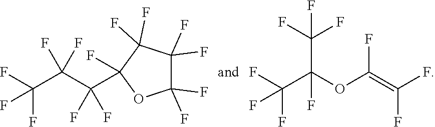 [SMILES: FC(F)(F)C(F)(F)C(F)(F)C1(F)OC(F)(F)C(F)(F)C1(F)F ; FC(F)=C(F)OC(F)(C(F)(F)F)C(F)(F)F]

In a certain embodiment, the component A is selected from one or two of

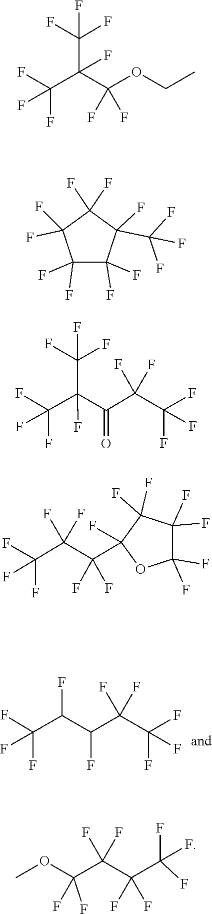

In a certain embodiment, the component B is preferably selected from a C₃ bromofluoroalkene and/or a C₄ iodoperfluoroalkane; for example, selected from one or more of

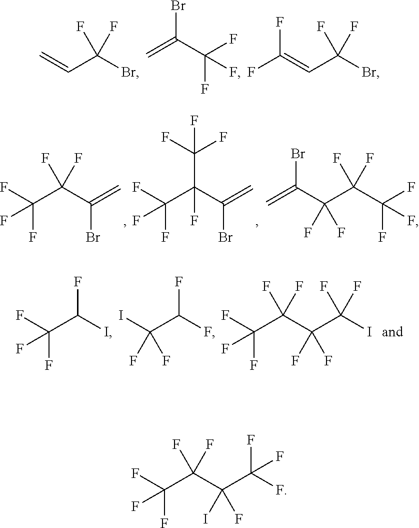

In a certain embodiment, the component B is more preferably selected from

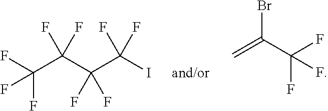

In a certain embodiment, the boiling point of the component A is preferably 25-75° C., such as 48° C., 49.2° C., 49.5° C., 55° C., 60° C. or 72° C.

In a certain embodiment, the boiling point of the component B is preferably 30-67° C., such as 34° C. or 64-67° C.

In a certain embodiment, the molecular weight of the component A is preferably 250-370 g/mol, such as 250 g/mol, 252 g/mol, 366 g/mol, 264 g/mol, 300 g/mol or 316 g/mol.

In a certain embodiment, the molecular weight of the component B is preferably 170-350 g/mol, such as 175 g/mol or 346 g/mol.

In a certain embodiment, the latent heat of vaporization of the component A is preferably 26-32 kJ/mol, such as 27.3 kJ/mol, 27.8 kJ/mol, 27.9 kJ/mol, 28.3 kJ/mol, 30.7 kJ/mol or 31.5 kJ/mol.

In a certain embodiment, the latent heat of vaporization of the component B is preferably 28-33 kJ/mol, such as 30.6 kJ/mol or 29.6 kJ/mol.

In a certain embodiment, the safeguard agent adopts scheme 1, scheme 2, scheme 3, scheme 4, scheme 5, scheme 6, scheme 7 or scheme 8

| | | | Molar ratio of |
|---|---|---|---|
| | | | component A to |
| | Component A | Component B | component B |
| Scheme 1 | 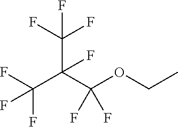 [SMILES: CCOC(F)(F)C(F)(C(F)(F)F)C(F)(F)F] | 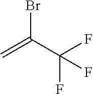 | 1:3 |
| Scheme 2 | 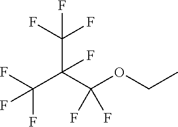 [SMILES: CCOC(F)(F)C(F)(C(F)(F)F)C(F)(F)F] | 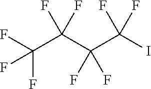 [SMILES: FC(F)(F)C(F)(F)C(F)(F)C(F)(F)I] | 1:2 |
| Scheme 3 | 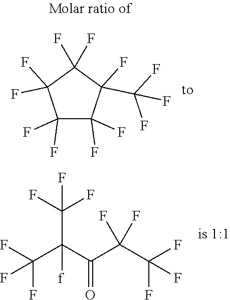 | 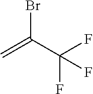 | 2:5 |
| Scheme 4 | 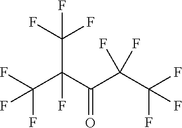 [SMILES: O=C(C(F)(F)C(F)(F)F)C(F)(C(F)(F)F)C(F)(F)F] | 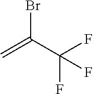 | 1:3 |
| Scheme 5 | 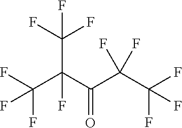 [SMILES: O=C(C(F)(F)C(F)(F)F)C(F)(C(F)(F)F)C(F)(F)F] | 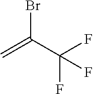 | 1:2 |
| Scheme 6 | 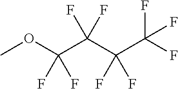 | 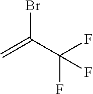 | 1:4 |
| Scheme 7 | 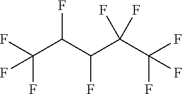 [SMILES: FC(C(F)C(F)(F)C(F)(F)F)C(F)(F)F] | 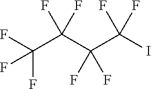 [SMILES: FC(F)(F)C(F)(F)C(F)(F)C(F)(F)I] | 1:1 |
| Scheme 8 | 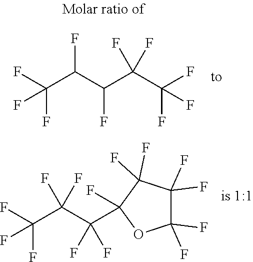 | 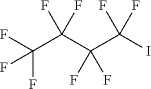 [SMILES: FC(F)(F)C(F)(F)C(F)(F)C(F)(F)I] | |

The present disclosure also provides a thermal runaway suppressant or a fire suppressant comprising a mixture of the above-mentioned safeguard agent and one or more extinguishants.

The present disclosure also provides an extinguishing equipment comprising the above-mentioned safeguard agent or the above-mentioned thermal runaway suppressant or the fire suppressant.

The present disclosure also provides a battery system comprising the above-mentioned safeguard agent or the above-mentioned thermal runaway suppressant or the fire suppressant, wherein the safeguard agent or the thermal runaway suppressant or fire suppressant is used to suppress or terminate thermal runaway of the battery system.

The present disclosure also provides a safeguard agent system comprising one or more safeguard agent containers;
when the safeguard agent system comprises a plurality of the containers, each container independently contains the above-mentioned component A and/or the above-mentioned component B, respectively stored in the containers; the molar ratio of the component A to the component B in the containers is 1:(1-4);
when the safeguard agent system comprises one container, the above-mentioned component A and the component B are stored in the container at a molar ratio of 1:(1-4).

In a certain embodiment, the safeguard agent system plays the role of cooling, isolation, antiflaming, explosion suppression or extinguishing by releasing the safeguard agent. When the safeguard agent system comprises two or more containers, the agents in the two or more containers can be released simultaneously or sequentially, if satisfied, the molar ratio of component A to component B in the released agents is 1:(1-4), preferably 1:(2-4), such as 1:3. The present disclosure also provides a method for controlling thermal runaway comprising the following steps: releasing or releasing in advance the above-mentioned safeguard agent to an object that has appeared thermal runaway or an object that is about to appear thermal runaway;

alternatively, releasing or releasing in advance the above-mentioned component A and the component B respectively to the object that has appeared thermal runaway or the object that is about to appear thermal runaway.

The molar rate ratio of the release of the component A to the component B is 1:(1-4), preferably 1:(2-4), such as 1:3.

In the method, the method can be used in a walled space, a sealed space or an enclosure, such as the walled space or the enclosure.

In the method, the number of the release of the safeguard agent can be one or more times.

In the method, the more times are preferably three times.

In the method, the method is used for the object that has appeared thermal runaway or the object that is about to appear thermal runaway, and the object that has appeared thermal runaway or the object that is about to appear thermal runaway is a battery that release energy in seconds or combustibles that have characteristics of deep-seated fire such as wood, paper, cotton, and grain.

The battery is preferably a battery that releases energy in seconds when thermal runaway occurs, preferably a lithium-ion battery that releases energy in seconds.

The lithium-ion battery is a ternary lithium battery, a lithium-cobalt battery or a lithium manganese battery.

The lithium-ion battery is a prismatic battery, a pouch battery or a cylinder battery.

The release mode of energy release is violent heat generation, gas generation or burning; the thermal runaway is mostly caused by internal short circuit or external short circuit of the battery and the like, which can be simulatively induced through nail penetration or heating experiment.

### DEFINITION OF TERMS

The substituted perfluorinated compound in the present disclosure is that the fluorine atoms in the compound are substituted by other groups, for example, the C₂-C₆ perfluorocyclic ether substituted by one or more C₁-C₃ perfluoroalkyl groups is that the fluorine atoms on the C₂-C₆ perfluorocyclic ether are substituted by one or more C₁-C₃ perfluoroalkyl groups.

In the present disclosure, the term “more” is 2, 3, 4 or more.

In the present disclosure, energy release in seconds refers to a state of violent energy release when thermal runaway occurs, and the duration is within 10 seconds, 30 seconds or 60 seconds, and the releasing duration is within about 100 seconds when the cell energy is large.

In the present disclosure, a sealed space refers to a tightly closed and well-sealed space to prevent water vapor from entering or steam from evaporating. Its subordinate concepts include high IP level electrical cabinets and boxes, such as IP67 or IP68.

In the present disclosure, an enclosure refers to a closed but not sealed space to prevent dust or foreign objects from entering. Its subordinate concepts include fire gas protected closed spaces with less leakage and able to maintain gas fire extinguishing concentration; and low IP level electrical cabinets, such as indoor IP34, IP44 or outdoor IP54, IP56.

In the present disclosure, an walled space refers to a space enclosed on all sides, such as a space with no leakage on all sides, such as a space with no leakage on all sides and bottom.

The above preferred conditions can be combined arbitrarily to obtain preferred embodiments of the present disclosure without violating common knowledge in the art.

The reagents and raw materials used in the present disclosure are all commercially available.

The positive progressive effect of the present disclosure is that the safeguard agent comprised in the present disclosure can cool the protected space and heat sources, physically isolate heat sources, prevent burning or extinguish the flame in the space, suppress explosion, and effectively and continuously suppress and prevent reignition. Using the safeguard agent of the present disclosure as a suppression agent for battery thermal runaway can achieve the purpose of terminating the thermal runaway after cooling, antiflaming, suppression, or extinguishing of a battery in thermal runaway, and more batteries can achieve constant voltage and good appearance without damage phenomena. For other batteries affected by heat in the space, the safeguard agent can also effectively prevent or suppress potential thermal runaway.

### DETAILED DESCRIPTION OF THE INVENTION

The present disclosure will be further described below with reference to examples, but the present disclosure is not therefore limited to the scope of the examples. Experimental methods without specific conditions in the following examples are selected according to conventional methods and conditions, or according to the commercial specification.

The test scenes in the detailed description of the preferred embodiment are shown in the following table

| | | | Trigger | Steady- |
|---|---|---|---|---|
| NO. | Test box | Battery type | method | state time |
| Scene 1 | 1200 L cabinet | 21700 module 3 × 3 | Nail | Vigorous |
| | without top | cell 4.8 Ah 100% SOC (State | penetration | release 33 s |
| | | of Charge)(21700 refers to a | | |
| | | cylinder battery with a | | |
| | | diameter of 21 mm and a | | |
| | | length of 70 mm) | | |
| Scene 2 | 500 L enclosure | Ternary prismatic battery | Nail | Vigorous |
| | box | 40 Ah | penetration | release 13 s |
| | | A 100% SOC cell (prismatic | | |
| | | refers to a battery is a hard | | |
| | | shell cuboid) | | |
| Scene 3 | 150 L airtight | Ternary pouch battery 58 Ah | Electric | Vigorous |
| | box | A 100% SOC cell (pouch | heating | release 10 s |
| | | refers to a battery is a pouch- | | |
| | | shaped thin cuboid) | | |

Note:
1. A cabinet without top means that the top is completely open, the surrounding and bottom are steel plates, and there is no leakage around, simulating the environment of a battery warehouse or a battery cabinet with an open top, which is one of the walled spaces.

Enclosure box refers to a sealed but not airtight, less leakage, about IP44, simulating energy storage battery cabinet.

Airtight box simulates the airtight environment of the vehicle battery package, and has good airtightness, very little leakage, about IP67 or more.

Both airtight and enclosure test box are equipped with safety measures which are discontinuous relief device.

2. The violent release of thermal runaway of the battery cell refers to the violent release duration of the stored power of the battery cell. The violent release refers to the generation of high-speed off-gas, deflagration, and continuous mass fire. Small smoke and small fire are not violent.

3. The trigger method refers to the method of causing thermal runaway of the battery cell. Generally, nail penetration or electric heating is used to simulate the thermal runaway caused by an internal short circuit, so that the battery energy is released in seconds.

4. 100% SOC means that the battery is fully charged, and the Ah number is the battery capacity, indicating the power that can be stored.

IP is the level of protection against ingress of foreign objects by the casing of electrical equipment, as defined in the standard GB 4208-2008/IEC 60529-2001 “Degrees of Protection Provided by Enclosure (IP Code)”.

The burning situation of each test scene in the detailed description of the preferred embodiment when not adding safeguard agent is shown in the following table;

| | | Thermal | Steady- | |
|---|---|---|---|---|
| | Description of thermal runaway | runaway | state | |
| NO. | phenomenon | duration | duration | Battery test |
| Scene 1 | A cell is penetrated by a nail; | Deflagration | Vigorously | The voltage |
| | thermal runaway and deflagration | and burning | lasts 33 s | of these 9 |
| | occur, and the flame gradually | last for 1 min | | pcs cells are |
| | extinguishes; after 5 minutes | 16 s | | 0 |
| | without flame and with smoke, the | | | |
| | rest of the battery cells are | | | |
| | discontinuously thermal runaway | | | |
| | and deflagrated, and continued to jet | | | |
| | fire. | | | |
| Scene 2 | A large amount of gas and sparks are | Off-gas | Vigorously | The voltage |
| | generated, and an explosion occurs | jetting, | lasts for 13 s | is 0, the |
| | instantly, blowing up the test box | deflagration | | casing is |
| | and smashing the observation | and burning | | cracked and |
| | window. Deflagrates violently, jets | last for 28 s | | lifted |
| | fire and burns for a while. | | | |
| Scene 3 | The cell punch ruptures and catches | Burning and | Vigorously | The voltage |
| | fire. The fire continues to be jets, | deflagration | lasts for 10 s | is 0, and |
| | and the pressure relief plate bounces | last for 15 s, | | there are |
| | with time intervals. After there is no | and live coal- | | many splits |
| | open flame, a large amount of | like flameless | | around |
| | smoke is still produced. | burning lasts | | |
| | | for a long | | |
| | | time | | |

Each component name and structure in the detailed description of the preferred embodiment are shown in the following table:

| | | Latent heat of | | |
|---|---|---|---|---|
| Component | | vaporization | Boiling point | Molecular |
| name | Structure | KJ/mol | ° C. | weight g/mol |
| A1 | 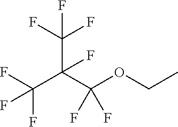 [SMILES: CCOC(F)(F)C(F)(C(F)(F)F)C(F)(F)F] | 27.3 KJ/mol | 49.5 | 264 |
| A2 | 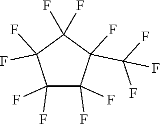 [SMILES: FC(F)(F)C1(F)C(F)(F)C(F)(F)C(F)(F)C1(F)F] | 27.9 KJ/mol | 48 | 300 |
| A3 | 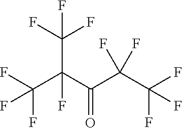 [SMILES: O=C(C(F)(F)C(F)(F)F)C(F)(C(F)(F)F)C(F)(F)F] | 27.8 KJ/mol | 49.2 | 316 |
| A4 | 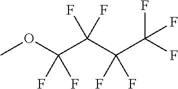 | 28.3 KJ/mol | 60 | 250 |
| A5 | 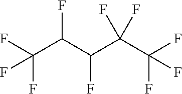 [SMILES: FC(C(F)C(F)(F)C(F)(F)F)C(F)(F)F] | 30.7 KJ/mol | 55 | 252 |
| A6 | 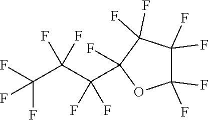 [SMILES: FC(F)(F)C(F)(F)C(F)(F)C1(F)OC(F)(F)C(F)(F)C1(F)F] | 31.5 KJ/mol | 72 | 366 |
| B1 | 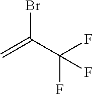 | 30.6 KJ/mol | 34 | 175 |
| B2 | 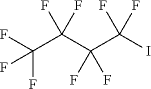 [SMILES: FC(F)(F)C(F)(F)C(F)(F)C(F)(F)I] | 29.6 KJ/mol | 64-67 | 346 |

The component A and the component B were mixed homogeneously according to a certain volume ratio. Where, the types and proportions of the component A and component B are specifically shown in the following table:

| | | | Molar ratio of |
|---|---|---|---|
| Example | | | component A to |
| number | Component A | Component B | component B |
| Example 1 | A1 | B1 | A1:B1 = 1:3 |
| Example 2 | A1 | B2 | A1:B2 = 1:2 |
| Example 3 | A2 + A3 | B1 | A2:A3:B1 = 1:1:5 |
| Example 4 | A3 | B1 | A3:B1 = 1:3 |
| Example 5 | A3 | B1 | A3:B1 = 1:2 |
| Example 6 | A4 | B1 | A4:B1 = 1:4 |
| Example 7 | A5 | B2 | A5:B2 = 1:1 |
| Example 8 | A5 + A6 | B2 | A5:A6:B1 = 1:1:4 |

### Mixing Method of Agent

Component A and component B were mixed and stirred evenly to obtain the safeguard agent.

Releasing Method of Agent

The safeguard agent was stored in a container, pressurized by the gas, and when released, the pressure pushed the safeguard agent to release through a pipeline and a nozzle.

At this time, the gas could be an inert gas such as N₂ or Ar₂.

The safeguard agent could be sucked or pushed out by a method of a pump or a piston, and released through a pipeline and a nozzle.

Effect Example 1: The effect data of the safeguard agent in Example 1 for scene 1, 2 or 3

| | Suppression mode and | Agent | Unburned | |
|---|---|---|---|---|
| NO. | phenomenon description | dose g | duration | Battery test |
| Scene 1 | The agent is released totally, and | 1300 | It is observed that | The voltage of |
| | then a cell is penetrated. It can be | | the unburned | one cell is 0 V, |
| | seen that sparks are ejected, but no | | duration is 10 | the voltage of |
| | flame is seen. It can be seen that | | minutes, and the | the adjacent one |
| | the casing of the penetrated cell | | burning torch in | is 2.6 V, and the |
| | turns red, and no burning is seen | | the test is still | remaining 7 pcs |
| | until the end. | | extinguished | are available at |
| | | | | 4.2 V. |
| Scene 2 | The agent is sprayed once in | 2100 | It is observed that | The voltage is 0 |
| | advance, then the cell is | | the unburned | V, the cell film |
| | penetrated, and then the agent is | | duration is 90 | isn't melted; and |
| | released automatically with time | | minutes, and the | the bottom film |
| | intervals. The cell generates a | | burning torch is | is relatively |
| | large amount of off-gas with | | extinguished in | intact |
| | violent disturbance, and gas | | three tests | |
| | overflows from the gap in the box, | | | |
| | sparks can be seen, and no | | | |
| | burning is seen until the end. | | | |
| Scene 3 | The film punch ruptures and jets | 900 | It is observed that | The voltage is 0 |
| | fire, and the agent is released | | the unburned | V, and the |
| | automatically with time intervals | | duration is 90 min, | battery is |
| | for multiple times. It can be seen | | and the burning | seriously |
| | that the flame lasts for about 2 | | torch is | cracked with |
| | seconds. After the fire is | | extinguished in | obvious splits. |
| | extinguished, there is still a high- | | three tests | |
| | speed off-gas. It can be seen that | | | |
| | the interior is like live coal, and | | | |
| | the pressure relief plate bounces | | | |
| | with time intervals. The | | | |
| | disturbance in the box gradually | | | |
| | calms down, and and no burning | | | |
| | is seen until the end. | | | |

Effect Example 2: The effect data of the safeguard agent in Example 2 for scene 1, 2 or 3

| | Suppression mode and | Agent | Unburned | |
|---|---|---|---|---|
| NO. | phenomenon description | dose g | duration | Battery test |
| Scene 1 | The agent is sprayed once in | 2100 | It is observed that | The voltage of |
| | advance, then the cell is | | the unburned | one cell is 0 V, |
| | penetrated. It can be seen that | | duration is 15 | the voltage of |
| | sparks are ejected, but no flame | | minutes, and the | the adjacent one |
| | is seen. It can be seen that the | | burning torch in | is 1.4 V, and the |
| | casing of the penetrated cell turns | | the test is still | remaining 7 pcs |
| | red, and no burning is seen until | | extinguished | are available at |
| | the end. | | | 4.2 V. |
| Scene 2 | When the cell is penetrated, the | 3400 | It is observed that | The voltage is 0 |
| | agent is released automatically | | the unburned | V, the cell film |
| | with time intervals for multiple | | duration is 110 | isn't melted; and |
| | times. The cell generates a large | | minutes, and the | the bottom film |
| | amount of off-gas with violent | | burning torch is | is relatively |
| | disturbance, and gas overflows | | extinguished in | intact |
| | from the gap in the box, sparks | | three tests | |
| | can be seen, and no burning is | | | |
| | seen until the end. | | | |
| Scene 3 | The film punch ruptures and jets | 1500 | It is observed that | The voltage is 0 |
| | fire, and the agent is released | | the unburned | V, the battery |
| | totally. It can be seen that the | | duration is 110 | has obvious |
| | flame lasts for about 1 second. | | min, and the | splits. |
| | After the fire is extinguished, | | burning torch is | |
| | there is still a high-speed off-gas. | | extinguished in | |
| | It can be seen that the interior is | | three tests | |
| | like live coal, and the pressure | | | |
| | relief plate bounces in the early | | | |
| | stage. The disturbance in the box | | | |
| | gradually calms down, and no | | | |
| | burning is seen until the end. | | | |

Effect Example 3: The effect data of the safeguard agent in Example 3 for scene 1, 2 or 3

| | Suppression mode and | Agent | Unburned | |
|---|---|---|---|---|
| NO. | phenomenon description | dose g | duration | Battery test |
| Scene 1 | The agent is released totally, | 1400 | It is observed that | The voltage of one |
| | and then a cell is penetrated. It | | the unburned | cell is 0 V, the |
| | can be seen that sparks are | | duration is 10 | voltage of the |
| | ejected, but no flame is seen. It | | minutes, and the | adjacent one is 1.8 |
| | can be seen that the casing of | | burning torch in | V, and the |
| | the penetrated cell turns red, | | the test is still | remaining 7 pcs are |
| | and no burning is seen until the | | extinguished | available at 4.2 V. |
| | end. | | | |
| Scene 2 | The agent is sprayed once in | 2250 | It is observed that | The voltage is 0 V, |
| | advance, then the cell is | | the unburned | the shape does not |
| | penetrated, and then the agent is | | duration is 90 | change |
| | released automatically with | | min, and the | significantly, and |
| | time intervals. The cell | | burning torch is | the bottom cell |
| | generates a large amount of off- | | extinguished in | film is not melted |
| | gas with violent disturbance, | | three tests | |
| | and a small amount of gas | | | |
| | overflows from the gap in the | | | |
| | box, a few sparks can be seen, | | | |
| | and no flame is seen until the | | | |
| | end. | | | |
| Scene 3 | The cell punch ruptures and | 1000 | It is observed that | The voltage is 0 V, |
| | burns, and the agent is released | | the unburned | and the battery is |
| | automatically with time | | duration is 90 | seriously cracked |
| | intervals for multiple times. It | | min, and the | with obvious |
| | can be seen that the flame lasts | | burning torch is | splits. |
| | for about 3 seconds, and a high- | | extinguished in | |
| | speed off-gas is generated. It | | three tests | |
| | can be seen that the interior is | | | |
| | like live coal, and no flame is | | | |
| | seen. The pressure relief plate | | | |
| | bounces with time intervals. | | | |
| | The disturbance in the box | | | |
| | gradually calms down. | | | |

Effect Example 4: The effect data of the safeguard agent in Example 4 for scene 1, 2 or 3

| | Suppression mode and | Agent | Unburned | |
|---|---|---|---|---|
| NO. | phenomenon description | dose g | duration | Battery test |
| Scene 1 | A cell is penetrated, and the | 1400 | It is observed that | The voltage of one |
| | agent is released at the same | | the unburned | cell is 0 V, the |
| | time, which is sprayed | | duration is 10 | voltage of the |
| | multiple times. It can be seen | | minutes, and the | adjacent one is 0.18 |
| | that sparks are ejected, but no | | burning torch in | V, the voltage of the |
| | flame is seen. The casing of | | the test is still | other adjacent one is |
| | the penetrated cell turns red, | | extinguished | 0.26 V, and the |
| | and no burning is seen until | | | remaining 6 pcs are |
| | the end. | | | available at 4.2 V. |
| Scene 2 | When the cell is penetrated, | 2200 | It is observed that | The voltage is 0 V, |
| | the agent is released | | the unburned | the shape does not |
| | automatically with time | | duration is 90 | change significantly, |
| | intervals for multiple times. | | minutes, and the | and the bottom cell |
| | The cell generates a large | | burning torch is | film is not melted |
| | amount of off-gas with violent | | extinguished in | |
| | disturbance, and a small | | three tests | |
| | amount of gas overflows from | | | |
| | the gap in the box, a few | | | |
| | sparks can be seen, and no | | | |
| | burning is seen until the end. | | | |
| Scene 3 | The cell punch ruptures and | | | |
| | burns, and the agent is | | | |
| | released automatically with | 950 | It is observed that | The voltage is 0 V, |
| | time intervals for multiple | | the unburned | and the battery is |
| | times. It can be seen that the | | duration is 90 | seriously cracked |
| | flame lasts for about 3 | | min, and the | with obvious splits. |
| | seconds, and a high-speed off- | | burning torch is | |
| | gas is generated. It can be | | extinguished in | |
| | seen that the interior is like | | three tests | |
| | live coal, and no flame is | | | |
| | seen. The pressure relief plate | | | |
| | bounces with time intervals. | | | |
| | The disturbance in the box | | | |
| | gradually calms down. | | | |

Effect Example 5: The effect data of the safeguard agent in Example 5 for scene 1, 2 or 3

| | Suppression mode and | Agent | Unburned | |
|---|---|---|---|---|
| NO. | phenomenon description | dose g | duration | Battery test |
| Scene 1 | The agent is released totally, | 1500 | It is observed that | The voltage of one |
| | and then a cell is penetrated. It | | the unburned | cell is 0 V, the |
| | can be seen that sparks are | | duration is 10 | voltage of the |
| | ejected, but no flame is seen. | | minutes, and the | adjacent one is 2.1 |
| | The casing of the penetrated | | burning torch in | V, and the |
| | cell turns red, and no burning is | | the test is still | remaining 7 pcs |
| | seen until the end. | | extinguished | are available at 4.2 |
| | | | | V. |
| Scene 2 | The agent is sprayed once in | 2350 | It is observed that | The voltage is 0 V, |
| | advance, then the cell is | | the unburned | the shape does not |
| | penetrated, and then the agent is | | duration is 90 | change |
| | released automatically with | | min, and the | significantly, and |
| | time intervals. The cell | | burning torch is | the bottom cell |
| | generates a large amount of off- | | extinguished in | film is not melted |
| | gas with violent disturbance, | | three tests | |
| | and a small amount of gas | | | |
| | overflows from the gap in the | | | |
| | box, a few sparks can be seen, | | | |
| | and no flame is seen until the | | | |
| | end. | | | |
| Scene 3 | The cell punch ruptures and | 1000 | It is observed that | The voltage is 0 V, |
| | burns, and the agent is released | | the unburned | and the battery is |
| | automatically with time | | duration is 90 | seriously cracked |
| | intervals for multiple times. It | | min, and the | with obvious |
| | can be seen that the flame lasts | | burning torch is | splits. |
| | for about 4 seconds, and a high- | | extinguished in | |
| | speed off-gas is generated. It | | three tests | |
| | can be seen that the interior is | | | |
| | like live coal, and no flame is | | | |
| | seen. The pressure relief plate | | | |
| | bounces with time intervals. | | | |
| | The disturbance in the box | | | |
| | gradually calms down. | | | |

Effect Example 6: The effect data of the safeguard agent in Example 6 for scene 1, 2 or 3

| | Suppression mode and | Agent | Unburned | |
|---|---|---|---|---|
| NO. | phenomenon description | dose g | duration | Battery test |
| Scene 1 | A cell is penetrated, and the | 1300 | It is observed that | The voltage of one |
| | agent is released at the same | | the unburned | cell is 0 V, the |
| | time, which is sprayed | | duration is 10 | voltage of the |
| | multiple times. It can be seen | | minutes, and the | adjacent one is 0.47 |
| | that sparks are ejected and the | | burning torch in | V, the voltage of the |
| | casing of the penetrated cell | | the test is still | other adjacent one is |
| | turns red, and no burning is | | extinguished | 1.36 V, and the |
| | seen until the end. | | | remaining 6 pcs are |
| | | | | available at 4.2 V. |
| Scene 2 | When the cell is penetrated, | 2000 | It is observed that | The voltage is 0 V, |
| | the agent is released | | the unburned | the shape does not |
| | automatically with time | | duration is 90 | change significantly, |
| | intervals for multiple times. | | minutes, and the | and the bottom cell |
| | The cell generates a large | | burning torch is | film is not melted |
| | amount of off-gas with violent | | extinguished in | |
| | disturbance, and a small | | three tests | |
| | amount of gas overflowes | | | |
| | from the gap in the box, a few | | | |
| | sparks can be seen, and no | | | |
| | burning is seen until the end. | | | |
| Scene 3 | The cell punch ruptures and | 850 | It is observed that | The voltage is 0 V, |
| | jets fire, and the agent is | | the unburned | and the battery has |
| | released totally. It can be seen | | duration is 90 | obvious splits. |
| | that the flame lasts for about 2 | | min, and the | |
| | seconds, and a high-speed off- | | burning torch is | |
| | gas is still generated after the | | extinguished in | |
| | fire is extinguished. It can be | | three tests | |
| | seen that the interior is like | | | |
| | live coal, and the pressure | | | |
| | relief plate bounces many | | | |
| | times in the early stage. The | | | |
| | disturbance in the box | | | |
| | gradually calms down, and no | | | |
| | burning is seen until the end. | | | |

Effect Example 7: The effect data of the safeguard agent in Example 7 for scene 1, 2 or 3

| | Suppression mode and | Agent | Unburned | |
|---|---|---|---|---|
| NO. | phenomenon description | dose g | duration | Battery test |
| Scene 1 | A cell is penetrated with a nail, | 2000 | It is observed that | The voltage of |
| | and appeared thermal runaway | | the unburned | one cell is 0 V, |
| | and deflagration. The agent is | | duration is 15 | the voltage of the |
| | released totally. The burning | | minutes, and the | adjacent three are |
| | disappears instantly and lasts for | | burning torch in | 0.39 V, 0.72 V |
| | 1 second. The casing of the | | the test is still | and 1.95 V, and |
| | penetrated cell turns red and | | extinguished | the remaining |
| | then gradually darkens, and no | | | 5 pcs are available |
| | burning is seen until the end. | | | at 4.2 V. |
| Scene 2 | After the cell is penetrated and | 3150 | It is observed that | The voltage is 0 |
| | deflagration is visible, the agent | | the unburned | V, the shape has |
| | is released automatically with | | duration is 120 | obvious splits, |
| | time intervals, and the burning | | minutes, and the | and the bottom |
| | lasts for 3 s and extinguishes. | | burning torch is | cell film is melted |
| | The cell generates a large | | extinguished in | |
| | amount of off-gas with violent | | three tests | |
| | disturbance, and gas overflows | | | |
| | from the gap in the box, and no | | | |
| | burning is seen until the end. | | | |
| Scene 3 | When the cell punch ruptures, | | | |
| | the agent is released | 1350 | It is observed that | The voltage is 0 |
| | automatically with time intervals | | the unburned | V, and the battery |
| | for multiple times. The sparks | | duration is 120 | has obvious |
| | are ejected, and no burning is | | min, and the | splits. |
| | seen. A high-speed off-gas is | | burning torch is | |
| | generated. It can be seen that | | extinguished in | |
| | the interior is like live coal, and | | three tests | |
| | the pressure relief plate bounces | | | |
| | frequently in the early stage. | | | |
| | The disturbance in the box | | | |
| | gradually calms down. No | | | |
| | burning is seen until the end. | | | |

Effect Example 8: The effect data of the safeguard agent in Example 8 for scene 1, 2 or 3

| | Suppression mode and | Agent | Unburned | |
|---|---|---|---|---|
| NO. | phenomenon description | dose g | duration | Battery test |
| Scene 1 | A cell is penetrated, and the | 2200 | It is observed that | The voltage of one |
| | agent is released at the same | | the unburned | cell is 0 V, the |
| | time, which is sprayed multiple | | duration is 15 | voltage of the |
| | times. It can be seen that sparks | | minutes, and the | adjacent one is 1.2 |
| | are ejected, but no flame is | | burning torch in | V, and the |
| | seen. It can be seen that the | | the test is still | remaining 7 pcs are |
| | casing of the penetrated cell | | extinguished | available at 4.2 V. |
| | turns red, and no burning is | | | |
| | seen until the end. | | | |
| Scene 2 | When the cell is penetrated, the | 3500 | It is observed that | The voltage is 0 V, |
| | agent is released automatically | | the unburned | the shape does not |
| | with time intervals for multiple | | duration is 120 | change |
| | times, and a few sparks can be | | minutes, and the | significantly, and |
| | seen. The cell generates a large | | burning torch is | the bottom cell |
| | amount of off-gas with violent | | extinguished in | film is not melted |
| | disturbance, and gas overflows | | three tests | |
| | from the gap in the box, and no | | | |
| | burning is seen until the end. | | | |
| Scene 3 | When the cell is about to | 1500 | It is observed that | The voltage is 0 V, |
| | rupture due to serious swelling, | | the unburned | and the battery has |
| | the agent is sprayed in advance, | | duration is 120 | obvious splits. |
| | and then the agent is released | | minutes, and the | |
| | automatically with time | | burning torch is | |
| | intervals. Sparks are ejected, | | extinguished in | |
| | no burning is seen, and high- | | three tests | |
| | speed off-gas is generated, and | | | |
| | it can be seen the interior is like | | | |
| | live coal. The pressure relief | | | |
| | plate bounces frequently in the | | | |
| | early stage. The disturbance in | | | |
| | the box gradually calms down. | | | |
| | No burning is seen until the | | | |
| | end. | | | |

What is claimed is:

1. A safeguard agent, consisting of component A and component B; the component A is selected from one or more of a C₅-C₈ perfluoroalkane, a C₅-C₇ perfluorocycloalkane which is unsubstituted or substituted by one or more trifluoromethyl groups, a C₅-C₈ fluoroalkane containing 1, 2, 3 or 4 hydrogen atoms, a C₄-C₇ perfluoroketone, a C₄-C₇ fluoroether, a C₂-C₆ perfluorocyclic ether which is unsubstituted or substituted by one or more C₁-C₃ perfluoroalkyl groups and heptafluoro-2-(1,2,2-trifluoroethyleneoxy)propane; the C₄-C₇ fluoroether contains at least 7 fluorine atoms;
the component B is a C₃-C₅ bromofluoroalkene and/or a C₂-C₄ iodofluoroalkane; the bromofluoroalkene contains at least 2 fluorine atoms; the C₂-C₄ iodofluoroalkane contains at least 4 fluorine atoms;
the molar ratio of the component A to the component B is 1:(1-4).

2. The safeguard agent according to claim 1, wherein the safeguard agent satisfies one or more following conditions:
(1) the safeguard agent is a cooling suppressant or an extinguishant;
(2) the safeguard agent is used in a walled space, a sealed space or an enclosure, such as the walled space or the enclosure;
(3) the safeguard agent is used to protect a battery or suppress energy that is continuously released from the battery during thermal runaway;
(4) the molar ratio of the component A to the component B is 1:(2-4), such as 1:3;
(5) the C₂-C₄ iodofluoroalkane contains one iodine atom;
(6) the C₃-C₅ bromofluoroalkene contains one bromine atom;
(7) the C₅-C₈ perfluoroalkane is selected from perfluorohexane and/or perfluoroheptane;
(8) the C₅-C₈ fluoroalkane containing 1, 2, 3 or 4 hydrogen atoms is decafluoropentane and/or monohydrotridecafluorohexane;
(9) the C₅-C₇ perfluorocycloalkane which is unsubstituted or substituted by one or more trifluoromethyl groups is dodecafluorocyclohexane and/or perfluoromethylcyclopentane;
(10) the C₄-C₇ perfluoroketone contains 1 or 2 carbonyl groups;
(11) the C₄-C₇ fluoroether contains 1 or 2 oxygen atoms;
(12) the C₂-C₆ perfluorocyclic ether which is unsubstituted or substituted by one or more C₁-C₃ perfluoroalkyl groups contains one oxygen atom;
(13) the C₃-C₅ bromofluoroalkene is selected from one or more of 3-bromo-3,3-difluoropropene, 2-bromo-3,3,3-trifluoropropene, 3-bromo-1,1,3,3-tetrafluoropropene, 2-bromo-3,3,4,4,4-pentafluoro-1-butene, 2-bromo-3,4,4,4-tetrafluoro-3-(trifluoromethyl)but-1-ene and 2-bromo-3,3,4,4,5,5,5-octafluoro-1-pentene;
(14) the C₂-C₄ iodofluoroalkane is selected from one or more of 1,1,1,2-tetrafluoro-2-iodoethane, iodo-1,1,2,2-tetrafluoroethane and iodoperfluorobutane;
(15) the boiling point of the component A is 20-85° C.;
(16) the boiling point of the component B is 30-80° C.;
(17) the molecular weight of the component A is 200-400 g/mol;
(18) the molecular weight of the component B is 150-400 g/mol;
(19) the latent heat of vaporization of the component A is 21.6-34.4 kJ/mol;
and (20) the latent heat of vaporization of the component B is 24.2-33.2 kJ/mol.

3. The safeguard agent according to claim 2, wherein the safeguard agent satisfies one or more following conditions:
(1) the battery is a battery that releases energy in seconds when thermal runaway occurs;
(2) the perfluorohexane is perfluoro-n-hexane, perfluoro-2-methylpentane or perfluoro-2,3-dimethylbutane;
(3) the perfluoroheptane is perfluoro-n-heptane;
(4) the C₅-C₈ fluoroalkane containing 1, 2, 3 or 4 hydrogen atoms is 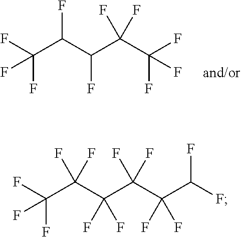 [SMILES: FC(C(F)C(F)(F)C(F)(F)F)C(F)(F)F ; FC(F)C(F)(F)C(F)(F)C(F)(F)C(F)(F)C(F)(F)F]
(5) the C₄-C₇ perfluoroketone is 1,1,1,3,4,4,4-heptafluoro-3-(trifluoromethyl)butan-2-one and/or perfluoro-2-methyl-3-pentanone;
(6) the C₄-C₇ fluoroether is selected from one or more of 2-(difluoro(methoxy)methyl)-1,1,1,2,3,3,3-heptafluoropropane, 2,2,3,3,3-pentafluoropropyl difluoromethyl ether, methylnonafluorobutylether, heptafluoropropyl-1,2,2,2-tetrafluoroethyl ether, 1,1,1,2,2,3,3-heptafluoro-3-methoxypropane, 2-(difluoromethoxymethyl)-1, 1,1,2,3,3,3-heptafluoropropane and 1-ethoxy-1,1,2,3,3,3-hexafluoro-2-(trifluoromethyl)propane;
(7) the C₂-C₆ perfluorocyclic ether which is unsubstituted or substituted by one or more C₁-C₃ perfluoroalkyl groups is perfluorocycloheptyl ether or perfluoro-2-methyl-2,3-epoxypentane;
(8) the C₃-C₅ bromofluoroalkene is selected from one or more of 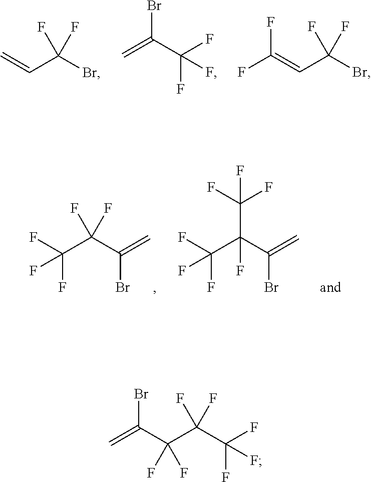
(9) the iodoperfluorobutane is 1-iodoperfluorobutane and/or 2-iodononafluorobutane;
(10) the boiling point of the component A is 25-75° C.;
(11) the boiling point of the component B is 30-67° C.;
(12) the molecular weight of the component A is 250-370 g/mol;
(14) the molecular weight of the component B is 170-350 g/mol;
(15) the latent heat of vaporization of the component A is 26-32 kJ/mol;
and (16) the latent heat of vaporization of the component B is 28-33 kJ/mol.

4. The safeguard agent according to claim 3, wherein the safeguard agent satisfies one or more following conditions:
(1) the battery is a lithium-ion battery that releases energy in seconds when thermal runaway occurs; the lithium-ion battery is preferably a ternary lithium battery, a lithium-cobalt battery or a lithium manganese battery; or the lithium-ion battery is a prismatic battery, a pouch battery or a cylinder battery;
(2) the release mode of energy release during thermal runaway is heat generation, gas generation, burning or explosion; the thermal runaway can be caused by internal short circuit or external short circuit of the battery, which can be simulatively induced through nail penetration or heating experiment;
(3) the C₄-C₇ fluoroether is selected from one or more of 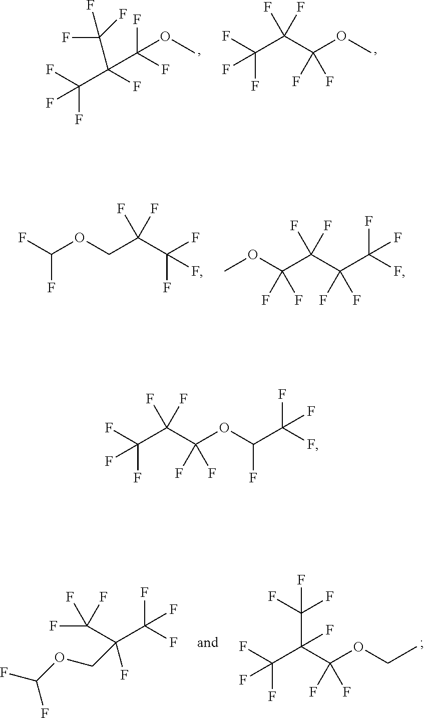
(4) the C₅-C₈ perfluoroalkane is selected from one or more of 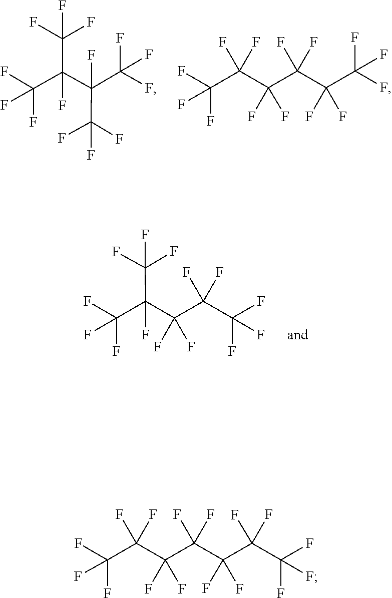
(5) the C₂-C₄ iodofluoroalkane is selected from one or more of 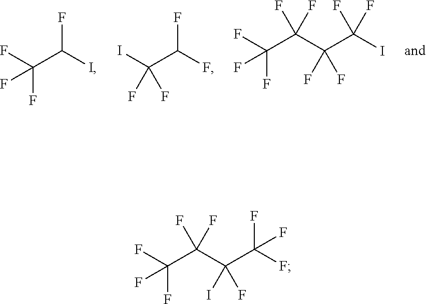
(6) the boiling point of the component A is 48° C., 55° C., 49.2° C., 49.5° C., 60° C. or 72° C.;
(7) the boiling point of the component B is 34° C. or 64-67° C.;
(8) the molecular weight of the component A is 250 g/mol, 252 g/mol, 366 g/mol, 264 g/mol, 300 g/mol or 316 g/mol;
(9) the molecular weight of the component B is 175 g/mol or 346 g/mol;
(10) the latent heat of vaporization of the component A is 27.3 kJ/mol, 27.8 kJ/mol, 27.9 kJ/mol, 28.3 kJ/mol, 30.7 kJ/mol or 31.5 kJ/mol;
and (11) the latent heat of vaporization of the component B is 30.6 kJ/mol or 29.6 kJ/mol.

5. The safeguard agent according to claim 1, wherein
(1) the safeguard agent is used to suppress heat generation, gas generation, burning or explosion caused by thermal runaway of a battery;
(2) the component A is selected from a C₅-C₇ perfluorocycloalkane which is unsubstituted or substituted by one or more trifluoromethyl groups, a C₄-C₇ perfluoroketone, a C₂-C₄ perfluorocyclic ether which is unsubstituted or substituted by one or more C₁-C₃ perfluoroalkyl groups and a C₄-C₇ fluoroether; the C₄-C₇ fluoroether contains at least 7 fluorine atoms;
(3) the component B is selected from a C₃ bromofluoroalkene and/or a C₄ iodoperfluoroalkane;
and (4) the safeguard agent is a homogeneous liquid.

6. The safeguard agent according to claim 1, wherein the safeguard agent satisfies one or two of the following conditions:
(1) the component A is selected from one or more of 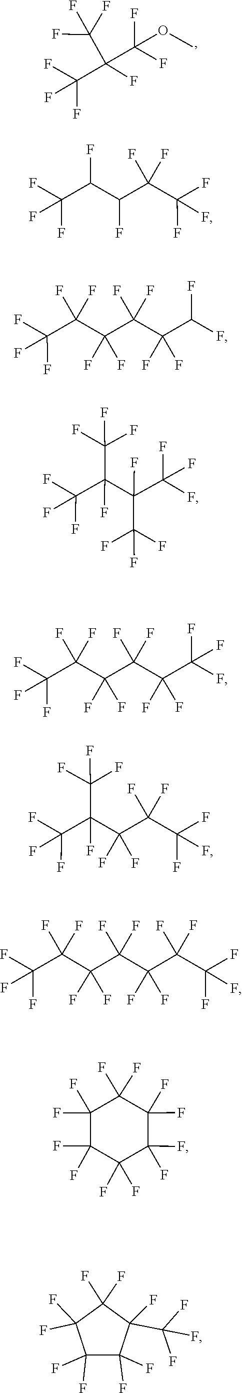 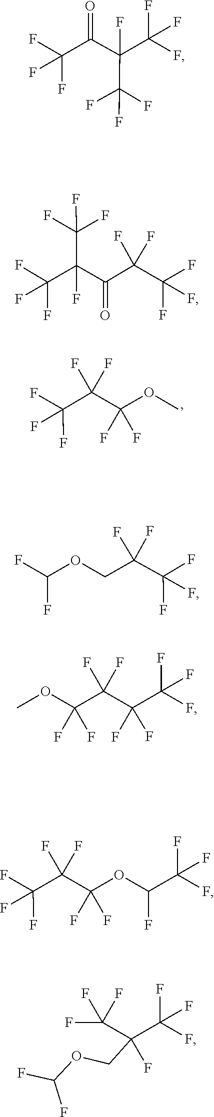 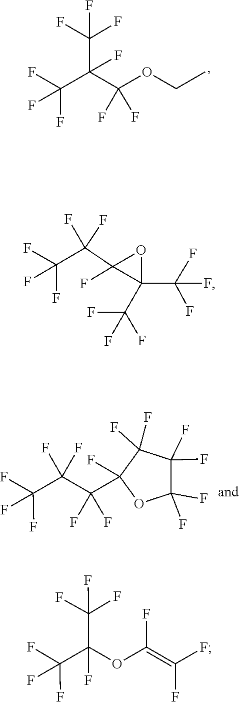 [SMILES: CCOC(F)(F)C(F)(C(F)(F)F)C(F)(F)F ; FC(F)(F)C(F)(F)C1(F)OC1(C(F)(F)F)C(F)(F)F ; FC(F)(F)C(F)(F)C(F)(F)C1(F)OC(F)(F)C(F)(F)C1(F)F ; FC(F)=C(F)OC(F)(C(F)(F)F)C(F)(F)F]
and (2) the component B is selected from one or more of 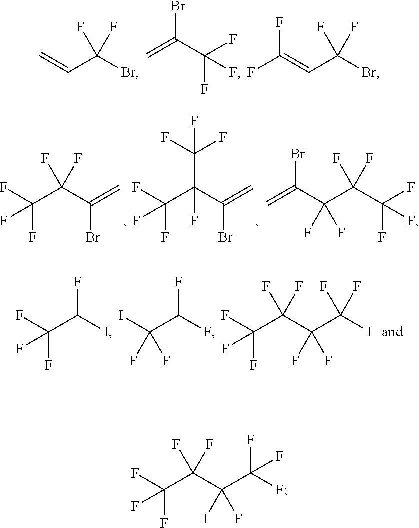
the safeguard agent preferably satisfies one or two of the following conditions:
(1) the component A is selected from one or two of 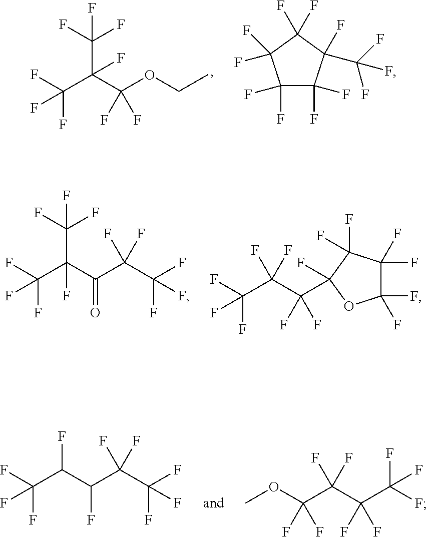
and (2) the component B is selected from

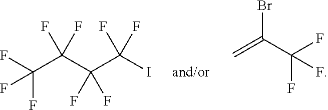

7. The safeguard agent according to claim 1, wherein the safeguard agent adopts scheme 1, scheme 2, scheme 3, scheme 4, scheme 5, scheme 6, scheme 7 or scheme 8

| | | | Molar ratio of |
|---|---|---|---|
| | | | component A to |
| | Component A | Component B | component B |
| Scheme 1 | 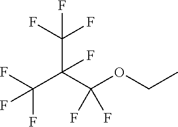 [SMILES: CCOC(F)(F)C(F)(C(F)(F)F)C(F)(F)F] | 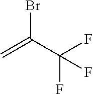 | 1:3 |
| Scheme 2 | 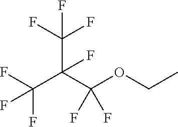 [SMILES: CCOC(F)(F)C(F)(C(F)(F)F)C(F)(F)F] | 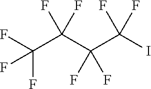 [SMILES: FC(F)(F)C(F)(F)C(F)(F)C(F)(F)I] | 1:2 |
| Scheme 3 | 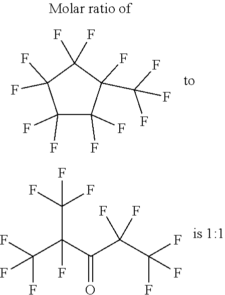 | 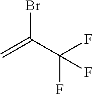 | 2:5 |
| Scheme 4 | 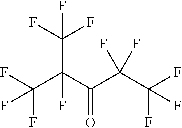 [SMILES: O=C(C(F)(F)C(F)(F)F)C(F)(C(F)(F)F)C(F)(F)F] | 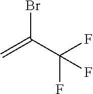 | 1:3 |
| Scheme 5 | 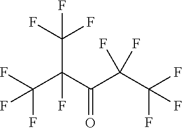 [SMILES: O=C(C(F)(F)C(F)(F)F)C(F)(C(F)(F)F)C(F)(F)F] | 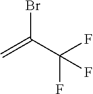 | 1:2 |
| Scheme 6 | 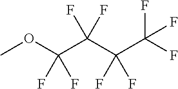 | 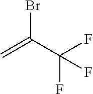 | 1:4 |
| Scheme 7 | 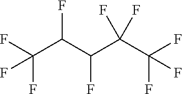 [SMILES: FC(C(F)C(F)(F)C(F)(F)F)C(F)(F)F] | 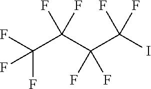 [SMILES: FC(F)(F)C(F)(F)C(F)(F)C(F)(F)I] | 1:1 |
| Scheme 8 | 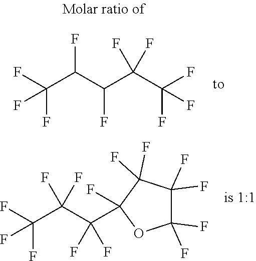 | 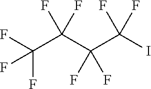 [SMILES: FC(F)(F)C(F)(F)C(F)(F)C(F)(F)I] | 1:2 |

8. A safeguard agent, consisting of component A and component B; and the latent heat of vaporization of the component A is 21.6-34.4 kJ/mol; the latent heat of vaporization of the component B is 24.2-33.2 kJ/mol; the boiling point of the component A is 20-85° C.; the boiling point of the component B is 30-80° C.; the number of fluorine atoms of the compound in the component A is 7-16, and the number of carbon atoms of the compound in the component A is 4-8; the compound in the component B contains at least one bromine or iodine atom; the number of hydrogen atoms of the compound in the component A is 0-5; the molecular weight of the component A is 200-400 g/mol; the molecular weight of the component B is 150-400 g/mol;
the molar ratio of the component A to the component B is 1:(1-4).

9. The safeguard agent according to claim 8, wherein the safeguard agent satisfies one or two of the following conditions:
(1) the component A is selected from one or more of a C₅-C₈ perfluoroalkane, a C₅-C₇ perfluorocycloalkane which is unsubstituted or substituted by one or more trifluoromethyl groups, a C₅-C₈ fluoroalkane containing 1, 2, 3 or 4 hydrogen atoms, a C₄-C₇ perfluoroketone, a C₄-C₇ fluoroether, a C₂-C₆ perfluorocyclic ether which is unsubstituted or substituted by one or more C₁-C₃ perfluoroalkyl groups and heptafluoro-2-(1,2,2-trifluoroethyleneoxy)propane; the C₄-C₇ fluoroether contains at least 7 fluorine atoms;
and (2) the component B is a C₃-C₅ bromofluoroalkene and/or a C₂-C₄ iodofluoroalkane; the bromofluoroalkene contains at least 2 fluorine atoms; the C₂-C₄ iodofluoroalkane contains at least 4 fluorine atoms.

10. The safeguard agent according to claim 9, wherein the safeguard agent satisfies one or more following conditions:
(1) the safeguard agent is a cooling suppressant or an extinguishant;
(2) the safeguard agent is used in a walled space, a sealed space or an enclosure, such as the walled space or the enclosure;
(3) the safeguard agent is used to protect a battery or suppress energy that continuously releases from the battery during thermal runaway;
(4) the molar ratio of the component A to the component B is 1:(2-4), such as 1:3;
(5) the C₂-C₄ iodofluoroalkane contains one iodine atom;
(6) the C₃-C₅ bromofluoroalkene contains one bromine atom;
(7) the C₅-C₈ perfluoroalkane is selected from perfluorohexane and/or perfluoroheptane;
(8) the C₅-C₈ fluoroalkane containing 1, 2, 3 or 4 hydrogen atoms is decafluoropentane and/or monohydrotridecafluorohexane;
(9) the C₅-C₇ perfluorocycloalkane which is unsubstituted or substituted by one or more trifluoromethyl groups is dodecafluorocyclohexane and/or perfluoromethylcyclopentane;
(10) the C₄-C₇ perfluoroketone contains 1 or 2 carbonyl groups;
(11) the C₄-C₇ fluoroether contains 1 or 2 oxygen atoms;
(12) the C₂-C₆ perfluorocyclic ether which is unsubstituted or substituted by one or more C₁-C₃ perfluoroalkyl groups contains one oxygen atom;
(13) the C₃-C₅ bromofluoroalkene is selected from one or more of 3-bromo-3,3-difluoropropene, 2-bromo-3,3,3-trifluoropropene, 3-bromo-1, 1,3,3-tetrafluoropropene, 2-bromo-3,3,4,4,4-pentafluoro-1-butene, 2-bromo-3,4,4,4-tetrafluoro-3-(trifluoromethyl)but-1-ene and 2-bromo-3,3,4,4,5,5,5-octafluoro-1-pentene;
and (14) the C₂-C₄ iodofluoroalkane is selected from one or more of 1,1,1,2-tetrafluoro-2-iodoethane, iodo-1,1,2,2-tetrafluoroethane and iodoperfluorobutane.

11. The safeguard agent according to claim 10, wherein the safeguard agent satisfies one or more following conditions:
(1) the battery is a battery that releases energy in seconds when thermal runaway occurs;
(2) the perfluorohexane is perfluoro-n-hexane, perfluoro-2-methylpentane or perfluoro-2,3-dimethylbutane;
(3) the perfluoroheptane is perfluoro-n-heptane; 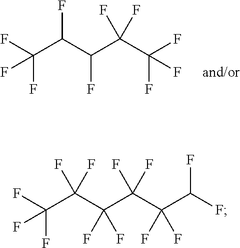 [SMILES: FC(C(F)C(F)(F)C(F)(F)F)C(F)(F)F ; FC(F)C(F)(F)C(F)(F)C(F)(F)C(F)(F)C(F)(F)F]
(4) the C₅-C₈ fluoroalkane containing 1, 2, 3 or 4 hydrogen atoms is
(5) the C₄-C₇ perfluoroketone is 1,1,1,3,4,4,4-heptafluoro-3-(trifluoromethyl)butan-2-one and/or perfluoro-2-methyl-3-pentanone;
(6) the C₄-C₇ fluoroether is selected from one or more of 2-(difluoro(methoxy)methyl)-1,1,1,2,3,3,3-heptafluoropropane, 2,2,3,3,3-pentafluoropropyl difluoromethyl ether, methylnonafluorobutylether, heptafluoropropyl-1,2,2,2-tetrafluoroethyl ether, 1,1,1,2,2,3,3-heptafluoro-3-methoxypropane, 2-(difluoromethoxymethyl)-1,1,1,2,3,3,3-heptafluoropropane and 1-ethoxy-1,1,2,3,3,3-hexafluoro-2-(trifluoromethyl)propane;
(7) the C₂-C₆ perfluorocyclic ether which is unsubstituted or substituted by one or more C₁-C₃ perfluoroalkyl groups is perfluorocycloheptyl ether or 2-methyl-2,3-epoxypentane;
(8) the C₃-C₅ bromofluoroalkene is selected from one or more of 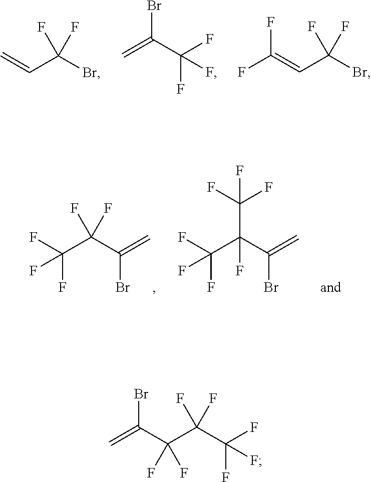
(9) the iodoperfluorobutane is 1-iodoperfluorobutane and/or 2-iodononafluorobutane;
(10) the boiling point of the component A is 25-60° C.;
(11) the boiling point of the component B is 30-67° C.;
(12) the molecular weight of the component A is 250-370 g/mol;
(14) the molecular weight of the component B is 170-350 g/mol;
(15) the latent heat of vaporization of the component A is 26-32 kJ/mol;
and (16) the latent heat of vaporization of the component B is 28-33 kJ/mol.

12. The safeguard agent according to claim 11, wherein the safeguard agent satisfies one or more following conditions:
(1) the battery is a lithium-ion battery that releases energy in seconds when thermal runaway occurs; the lithium-ion battery is preferably a ternary lithium battery, a lithium-cobalt battery or a lithium manganese battery; or the lithium-ion battery is a prismatic battery, a pouch battery or a cylinder battery;
(2) the release mode of energy release during thermal runaway is heat generation, gas generation, burning or explosion; the thermal runaway can be caused by internal short circuit or external short circuit of the battery, which can be simulatively induced through nail penetration or heating experiment;
(3) the C₄-C₇ fluoroether is selected from one or more of 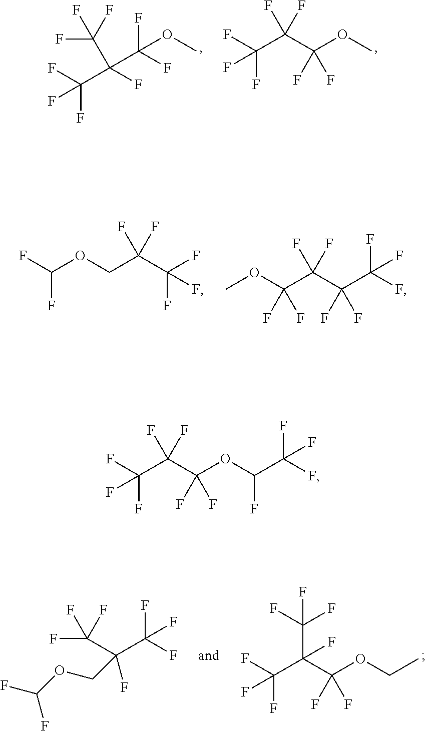
(4) the C₅-C₈ perfluoroalkane is selected from one or more of 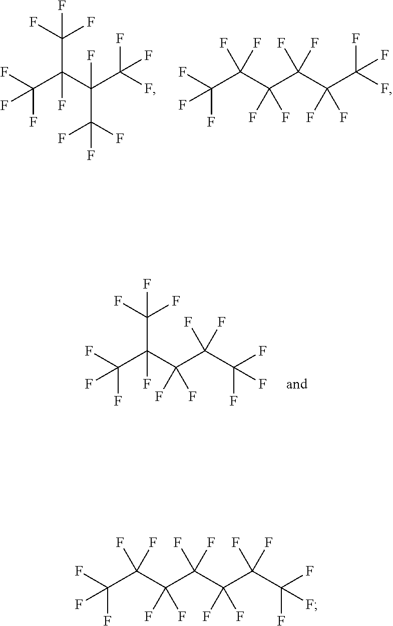
(5) the C₂-C₄ iodofluoroalkane is selected from one or more of 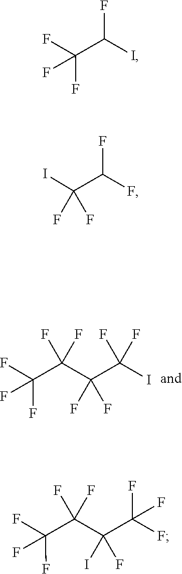 [SMILES: FC(I)C(F)(F)F ; FC(F)C(F)(F)I ; FC(F)(F)C(F)(F)C(F)(F)C(F)(F)I ; FC(F)(F)C(F)(F)C(F)(I)C(F)(F)F]
(6) the boiling point of the component A is 48° C., 49.2° C., 49.5° C., 55° C., 60° C. or 72° C.;
(7) the boiling point of the component B is 34° C. or 64-67° C.;
(8) the molecular weight of the component A is 250 g/mol, 252 g/mol, 366 g/mol, 264 g/mol, 300 g/mol or 316 g/mol;
(9) the molecular weight of the component B is 175 g/mol or 346 g/mol;
(10) the latent heat of vaporization of the component A is 27.3 kJ/mol, 27.8 kJ/mol, 27.9 kJ/mol, 28.3 kJ/mol, 30.7 kJ/mol or 31.5 kJ/mol;
and (11) the latent heat of vaporization of the component B is 30.6 kJ/mol or 29.6 kJ/mol.

13. The safeguard agent according to claim 8, wherein
(1) the safeguard agent is used to suppress heat generation, gas generation, burning or explosion caused by thermal runaway of a battery;
(2) the component A is selected from one or two of a C₅-C₇ perfluorocycloalkane which is unsubstituted or substituted by one or more trifluoromethyl groups, a C₄-C₇ perfluoroketone, a C₂-C₄ perfluorocyclic ether which is unsubstituted or substituted by one or more C₁-C₃ perfluoroalkyl and a C₄-C₇ fluoroether; the C₄-C₇ fluoroether contains at least 7 fluorine atoms;
(3) the component B is selected from a C₃ bromofluoroalkene and/or a C₄ iodoperfluoroalkane;
and (4) the safeguard agent is a homogeneous liquid.

14. The safeguard agent according to claim 8, wherein the safeguard agent satisfies one or two of the following conditions:
(1) the component A is selected from one or more of 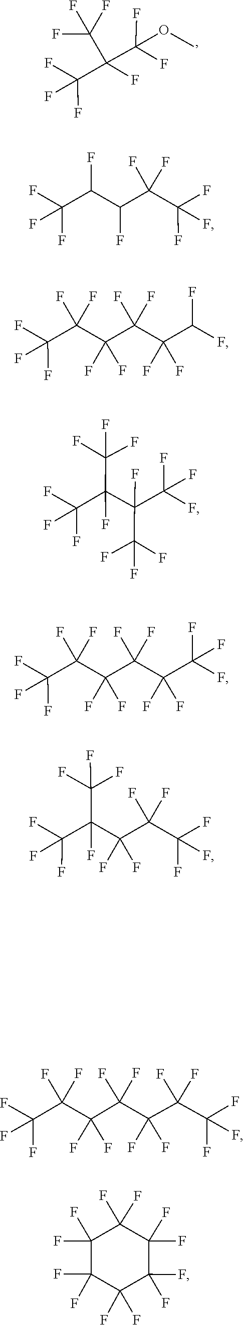 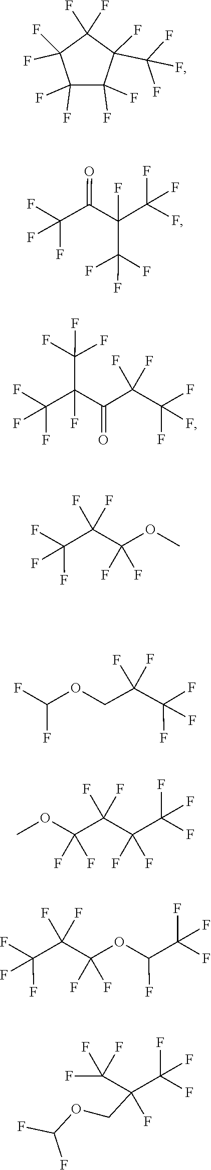 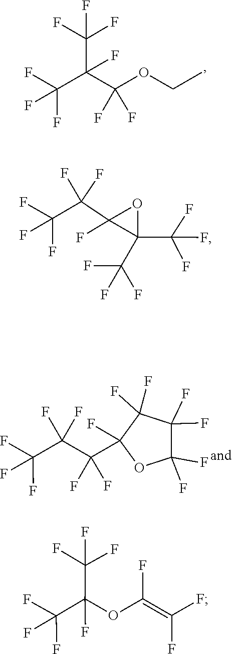 [SMILES: CCOC(F)(F)C(F)(C(F)(F)F)C(F)(F)F ; FC(F)(F)C(F)(F)C1(F)OC1(C(F)(F)F)C(F)(F)F ; FC(F)(F)C(F)(F)C(F)(F)C1(F)OC(F)(F)C(F)(F)C1(F)F ; FC(F)=C(F)OC(F)(C(F)(F)F)C(F)(F)F]
and (2) the component B is selected from one or more of 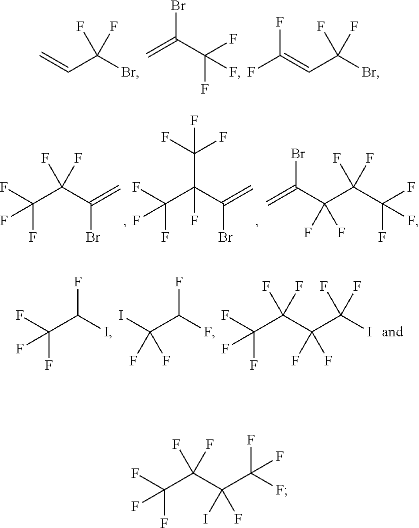
the safeguard agent preferably satisfies one or two of the following conditions:
(1) the component A is selected from one or two of; 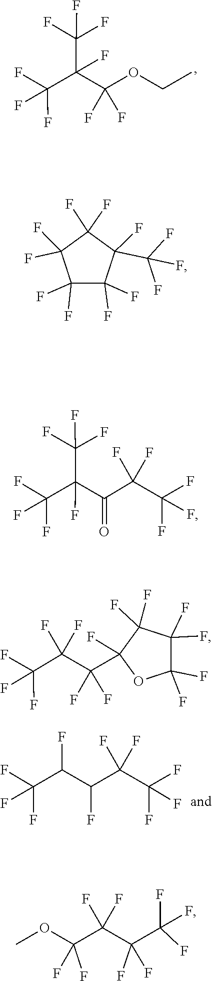
and (2) the component B is selected from

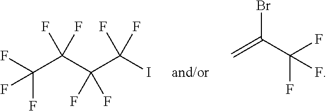

15. The safeguard agent according to claim 14, wherein the safeguard agent adopts scheme 1, scheme 2, scheme 3, scheme 4, scheme 5, scheme 6, scheme 7 or scheme 8

| | | | Molar ratio of |
|---|---|---|---|
| | | | component A to |
| | Component A | Component B | component B |
| Scheme 1 | 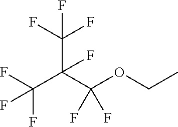 [SMILES: CCOC(F)(F)C(F)(C(F)(F)F)C(F)(F)F] | 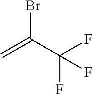 | 1:3 |
| Scheme 2 | 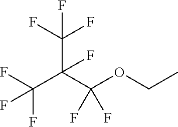 [SMILES: CCOC(F)(F)C(F)(C(F)(F)F)C(F)(F)F] | 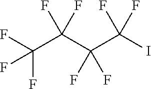 [SMILES: FC(F)(F)C(F)(F)C(F)(F)C(F)(F)I] | 1:2 |
| Scheme 3 | 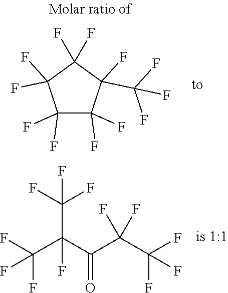 | 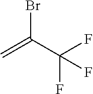 | 2:5 |
| Scheme 4 | 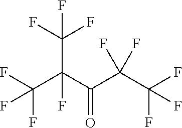 [SMILES: O=C(C(F)(F)C(F)(F)F)C(F)(C(F)(F)F)C(F)(F)F] | 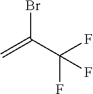 | 1:3 |
| Scheme 5 | 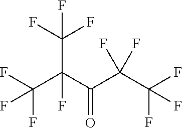 [SMILES: O=C(C(F)(F)C(F)(F)F)C(F)(C(F)(F)F)C(F)(F)F] | 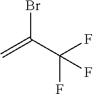 | 1:2 |
| Scheme 6 | 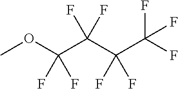 | 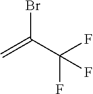 | 1:4 |
| Scheme 7 | 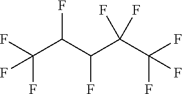 [SMILES: FC(C(F)C(F)(F)C(F)(F)F)C(F)(F)F] | 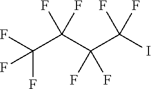 [SMILES: FC(F)(F)C(F)(F)C(F)(F)C(F)(F)I] | 1:1 |
| Scheme 8 | 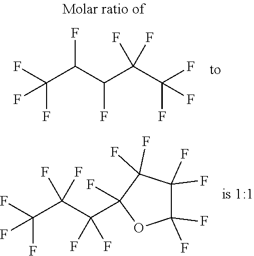 | 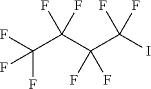 [SMILES: FC(F)(F)C(F)(F)C(F)(F)C(F)(F)I] | 1:2 |

16. A thermal runaway suppressant or a fire suppressant comprising a mixture of the safeguard agent according to claim 1 and one or more extinguishants.

17. An extinguishing equipment comprising the safeguard agent according to claim 1 or the thermal runaway suppressant or the fire suppressant comprising a mixture of the safeguard agent according to claim 1 and one or more extinguishants.

18. A battery system comprising the safeguard agent according to of claim 1 or the thermal runaway suppressant or the fire suppressant comprising a mixture of the safeguard agent according to claim 1 and one or more extinguishants.

19. A safeguard agent system comprising one or more safeguard agent containers;
when the safeguard agent system comprises a plurality of the containers, each container independently contains the component A according to claim 1 and/or the component B according to claim 1, respectively stored in the containers; the molar ratio of the component A to the component B in the containers is 1:(1-4);
when the safeguard agent system comprises one container, the component A and the component B according to claim 1 are stored in the container at a molar ratio of 1:(1-4).

20. A method for controlling thermal runaway comprising the following steps: releasing or releasing in advance the safeguard agent according to claim 1 to an object that has appeared thermal runaway or an object that is about to appear thermal runaway;
alternatively, releasing or releasing in advance the component A and the component B respectively according to claim 1 to the object that has appeared thermal runaway or the object that is about to appear thermal runaway, wherein the molar rate ratio of the release of the component A to the component B is preferably 1:(1-4), more preferably 1:(2-4), such as 1:3.
